(19) United States
(12) Patent Application Publication
(10) Pub. No.: US 2023/0167413 A1
(43) Pub. Date: Jun. 1, 2023

(54) ISOLATION AND FUNCTIONAL ANALYSIS OF EPITHELIAL PROGENITOR CELLS FROM THE HUMAN LUNG

(71) Applicant: CEDARS-SINAI MEDICAL CENTER, Los Angeles, CA (US)

(72) Inventors: Barry R. Stripp, Malibu, CA (US); Bindu Konda, Redondo Beach, CA (US); Apoorva Mulay, Los Angeles, CA (US)

(73) Assignee: CEDARS-SINAI MEDICAL CENTER, Los Angeles, CA (US)

(21) Appl. No.: 17/921,174

(22) PCT Filed: Apr. 30, 2021
(86) PCT No.: PCT/US2021/030280

§ 371 Date: Oct. 25, 2022

Related U.S. Application Data
(60) Provisional application No. 63/019,140, filed on May 1, 2020.
(60) Provisional application No. 63/019,217, filed on May 1, 2020.
(60) Provisional application No. 63/054,440, filed on Jul. 21, 2020.

(51) Int. Cl. C12N 5/071; G01N 33/50; G01N 1/30; G01N 33/569

(52) U.S. Cl. CPC C12N 5/0689; G01N 33/5044; G01N 1/30; G01N 33/56966; C12N 2513/00; C12N 2509/10; C12N 2501/727; C12N 2501/15

(57) ABSTRACT

The in vitro organoid model is a major technological breakthrough and an essential tool to study the basic biology of an organ system and for the development of various clinical applications for disease intervention. Organoids can self-renew and exhibit similarities in function as of their tissue of origin. Here, a step-by-step protocol is described to isolate region-specific progenitors from the human lung and generate 3D organoid cultures as an experimental and validation tool.

### CROSS-REFERENCE TO RELATED APPLICATIONS

This application includes a claim of priority under 35 U.S.C. § 119(e) to U.S. provisional patent applications No. 63/019,140 filed May 1, 2020, No. 63/019,217 filed May 1, 2020, and No. 63/054,440, filed Jul. 21, 2020, the entirety of all of which is hereby incorporated by reference.

### STATEMENT REGARDING FEDERALLY SPONSORED RESEARCH OR DEVELOPMENT

This invention was made with Government support under Grant No. HL108793 awarded by National Institutes of Health. The Government has certain rights in the invention.

### FIELD OF INVENTION

This invention relates to processing lung tissue and cells, and lung models.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Due to the challenges of consistently procuring fresh lung tissue from healthy donors, on-demand access to a stock of frozen tissues that faithfully recapitulate their native counterparts represents an invaluable asset to the pulmonary research community. The current state of the art for cryopreservation relating to the lung involves isolating cells from whole lung and freezing down all the cells for future experiment and to share collaborators and interested professionals. A lot of man power is involved in the processing of the lung along with expensive reagents, which takes up an enormous amount of working hours.

As such, there is a need in the art for new methods that address these issues while maintaining or improving the quality of frozen cells or tissues. The present invention addresses and solves these challenges.

Further, airspaces of the human respiratory system can be broadly divided into conducting and respiratory zones that mediate transport of gasses and their subsequent exchange across the epithelial-microvascular barrier, respectively. The conducting airways include the trachea, bronchi, bronchioles and terminal bronchioles whereas respiratory air spaces include the respiratory bronchioles, alveolar ducts and alveoli. The epithelial lining of these airspaces changes in composition along the proximo-distal axis to accommodate the unique requirements of each functionally distinct zone. The pseudostratified epithelium of tracheo-bronchial airways is composed of three major cell types, basal, secretory and ciliated, in addition to less abundant cell types including brush, neuroendocrine and ionocyte. Bronchiolar airways harbor morphologically similar epithelial cell types, although there are distinctions in their abundance and functional properties. For example, basal cells are less abundant within bronchiolar airways and secretory cells include a greater proportion of club cells versus serous and goblet cells that predominate in tracheobronchial airways. Epithelial cells of the respiratory zone include a poorly defined cuboidal cell type in respiratory bronchioles, in addition to alveolar type I (ATI) and type II (ATII) cells of alveolar ducts and alveoli.

The identity of epithelial stem and progenitor cell types that contribute to maintenance and renewal of epithelia in each zone are incompletely described and largely inferred from studies in animal models. Studies in mice have shown that either basal cells of pseudostratified airways, club cells of bronchiolar airways or ATII cells of the alveolar epithelium, serve as epithelial stem cells based upon capacity for unlimited self-renewal and multipotent differentiation. Despite the inability to perform genetic lineage tracing studies to assess sternness of human lung epithelial cell types, the availability of organoid-based culture models to assess the functional potential of epithelial stem and progenitor cells provides a tool for comparative studies between mouse and human. Herein the Inventors describe methods for isolation and functional analysis of epithelial cell types from different regions of the human lung.

Described herein are also tissue dissociation and cellular fractionation approaches allowing enrichment of epithelial cells from proximal and distal regions of the human lung. Herein these approaches are applied to the functional analysis of lung epithelial progenitor cells through use of 3D organoid culture models.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of processing and optionally freezing lung tissues, cells or both, comprising: cutting lung tissue into about 0.5 cm³ to 2.0 cm³ pieces; washing the pieces of tissue to remove blood, epithelial lining fluid, or both; optionally drying the pieces of lung tissue; removing visceral pleura from the pieces of lung tissue; and further cutting the pieces of lung tissue into about 0.5-5.0 mm diameter pieces, wherein the lung tissue is the proximal region of the lung or the distal region of the lung.

In various embodiments, the method can further comprise identifying and separating the proximal and distal regions before cutting the tissue into about 0.5 cm³ to 2.0 cm³ pieces.

In various embodiments, the method can comprise freezing the lung tissues, and the method can further comprise: placing the about 0.5-5.0 mm diameter pieces of lung tissue into a vial and cryoprotective media; and freezing the vial comprising the 0.5-5.0 mm diameter pieces of lung tissue and cryoprotective media to a temperature of about −90 to −70 degrees C.

In various embodiments, the method can further comprise freezing the 0.5-5.0 mm diameter pieces of lung tissue in vapor phase of a liquid nitrogen vessel.

In various embodiments, cutting lung tissue into about 0.5 cm³ to 2.0 cm³ pieces can comprise cutting lung tissue into about 1.0 cm³ pieces.

In various embodiments, further cutting the lung tissue into about 0.5-5.0 mm diameter pieces can comprise cutting the lung tissue into about 2-5 mm diameter pieces. In various embodiments, further cutting the lung tissue into about 0.5-5.0 mm diameter pieces can comprise cutting the lung tissue into about 3-4 mm diameter pieces.

In various embodiments, freezing the vial comprising the pieces of lung tissue to a temperature of about −90 to −70 degrees can comprise freezing the vial to about −80 degrees C.

Various embodiments of the present invention provide for a method of enrichment and optionally sub setting of small airway and aveolar epithelial progenitor cells from distal lung tissue, comprising: performing a method of processing and optionally freezing lung tissues, cells or both, as described above, wherein the lung tissue is distal lung tissue, and further cutting the lung tissue into about 0.5-5.0 mm diameter pieces comprises cutting the lung tissue into about 0.5-1.5 mm diameter pieces; digesting the about 0.5-1.5 mm diameter pieces of lung tissue with enzyme; dissociating the digested pieces of lung tissue into single cells; and selecting epithelial progenitor cells.

In various embodiments, cutting the lung tissue into about 0.5-1.5 mm diameter pieces can comprise first cutting the 0.5-5.0 mm diameter pieces of lung tissue into about 1.5-2.5 mm diameter pieces, and then cutting the 1.5-2.5 mm diameter pieces into about 0.5-1.5 mm diameter pieces.

In various embodiments, cutting the 1.5-2.5 mm diameter pieces into about 0.5-1.5 mm diameter pieces can comprise cutting the 1.5-2.5 mm diameter pieces into about 1.0 mm diameter pieces.

In various embodiments, the enzyme can comprise collagenase I, collagenase II, a non-clostridial neutral protease, or DNase, or combinations thereof.

In various embodiments, selecting the epithelial progenitor cells can comprise selecting cells having a surface marker profile that is one or more of: CD45-negative, CD31-negative, and CD236-positive, and optionally have a negative staining for DAPI.

In various embodiments, the method can comprise sub setting of small airway and aveolar epithelial progenitor cells, the method comprising: selecting epithelial cells that are HTII-280-negative as small airway epithelial progenitor cells; OR selecting epithelial cells are HTII-280-positive as alveolar type 2 (AT2) progenitor cells.

In various embodiments, selecting epithelial progenitor cells can comprise depleting immune cells and endothelial cells, cell surface staining for fluorescence associated cell sorting (FACS), or both.

Various embodiments of the present invention provide for a method of enrichment and optionally subsetting of epithelial progenitor cells from trachea-bronchial airways, comprising: performing a method performing a method of processing and optionally freezing lung tissues, cells or both, as described above, wherein the lung tissue is the proximal region of the lung, and wherein luminal epithelial cells have been removed from the proximal region of the lung; digesting the pieces of lung tissue with enzyme; dissociating the digested pieces of lung tissue into single cells; and selecting epithelial progenitor cells.

In various embodiments, the method can comprise: performing the following steps before performing a method of processing and optionally freezing lung tissues, cells or both, as described above: open airways of the distal lung tissue along their length to expose their lumen and cover the tissue with a solution comprising collagenase I, collagenase II, a non-clostridial neutral protease, or a combination thereof; stripping the luminal epithelial cells from the tissue; and collect the luminal epithelial cells.

In various embodiments, the method can comprise dissociating luminal epithelial cells into single cells.

In various embodiments, the method can further comprise combining the single cell luminal epithelial cells with the single cells before selecting for epithelial progenitor cells.

In various embodiments, the enzyme can comprise collagenase I, collagenase II, a non-clostridial neutral protease, or DNase, or combinations thereof.

In various embodiments, selecting the epithelial progenitor cells can comprise selecting cells having a surface marker profile that is one or more of: CD45-negative, CD31-negative, and CD236-positive, and optionally have a negative staining for DAPI.

In various embodiments the method can further comprising sub setting of epithelial progenitor cells, the method comprising: selecting epithelial progenitor cells that are NGRF-positive as a basal cell type; OR selecting epithelial progenitor cells that are NGRF-negative as a non-basal cell type.

In various embodiments selecting epithelial progenitor cells can comprise depleting immune cells and endothelial cells, cell surface staining for fluorescence associated cell sorting (FACS), or both.

Various embodiments of the provide for a method of generating a lung organoid, comprising: providing a quantity of cells isolated or produced by the methods described herein; culturing the cells in the presence of a growth media comprising a Rho kinase inhibitor; and further culturing the cells in the presence of a TGFβ inhibitor to generate lung organoids.

In various embodiments, the Rho kinase inhibitor can be Y-27632. In various embodiments, the TGFβ inhibitor can be SB431542. In various embodiments, the fluidic device can be a transwell system. In various embodiments, the fluidic device can be a microfluidic device. In various embodiments, culturing the cells can be for a period of about 7-40 days. In various embodiments, further culturing the cells can be for a period of about 15 days.

Various embodiments of the present invention provide for a quantity of lung organoids made by a method as described herein.

Various embodiments of the present invention provide for a system for modeling a lung, comprising: a population of cells comprising cells selected from the group consisting of lung cells isolated by any one of the methods described herein, primary lung cells differentiated from lung cells isolated by any one of the methods described herein, a lung organoid comprising the lung cells or the primary lung cells; a cell culture device, a cell culture plate, or a multi-well culture plate.

In various embodiments, the population of cells are in the cell culture device, the cell culture plate, or the multi-well culture plate.

Various embodiments provide for a system for test agent screening in a lung model, comprising: a population of cells comprising cells selected from the group consisting of lung cells isolated by any one of the methods as described herein, primary lung cells differentiated from lung cells isolated by any one of the methods described herein, a lung organoid comprising the lung cells or the primary lung cells; and a cell culture device, a cell culture plate, or a multi-well culture plate; wherein the test agent and the population of cells, are in contact in the cell culture plate, or the multi-well culture plate.

In various embodiments, cell culture device can be an air-liquid interface culture or a Transwell system comprising the population of cells.

In various embodiments, the lung cells can be epithelial cells. In various embodiments, the epithelial cells can be small airway epithelial progenitor cells, alveolar type 2 (AT2) progenitor cells, basal cell type, or non-basal cell type. In various embodiments, the epithelial cells can be proximal airway cells, or distal alveolar cells.

Various embodiments provide for a method selecting an agent of interest, comprising: contacting a test agent with a population of cells comprising cells selected from the group consisting of lung cells isolated by any one of the methods described herein, primary lung cells differentiated from lung cells isolated by any one of the methods of described herein, a lung organoid comprising the lung cells or the primary lung cells, wherein the test agent and the population of cells are in contact in a cell culture device, a cell culture plate, or a multi-well culture plate; measuring a parameter in the population of cells; and selecting the test agent as the agent of interest based on the measured parameter in the population of cells.

Various embodiments provide for a method modeling a lung, comprising: measuring a parameter in a population of cells comprising cells selected from the group consisting of lung cells isolated by any one of the methods described herein, primary lung cells differentiated from lung cells isolated by any one of the methods described herein, a lung organoid comprising the lung cells or the primary lung cells, wherein the population of cells are in contact in a cell culture device, a cell culture plate, or a multi-well culture plate.

In various embodiments, the method can further comprise contacting a test agent to the population of cells before, while, after or a combination thereof, measuring the parameter.

In various embodiments, the parameter comprises a phenotype of interest, an expression level of a gene, or an expression level of a protein of interest, or combination thereof.

In various embodiments, cell culture device is an air-liquid interface culture or a Transwell system comprising the population of cells.

In various embodiments, the lung cells are epithelial cells. In various embodiments, the epithelial cells are small airway epithelial progenitor cells, alveolar type 2 (AT2) progenitor cells, basal cell type, or non-basal cell type. In various embodiments, the epithelial cells are proximal airway cells, or distal alveolar cells.

In various embodiments, the lung cells are fibrotic lung cells. In various embodiments, the lung cells are idiopathic fibrotic lung cells.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels A-D) depicts an exemplary isolation strategy (A) Representative image of the human lung showing proximal and distal regions selected for cell isolation. (B) H&E staining of the proximal and distal regions of the lung. Proximal airways show a pseudostratified epithelium whereas the distal regions show presence of alveoli. (C, D) Immunofluorescent staining of corresponding regions showing NGFR⁺ basal progenitor cells at the basement membrane of the proximal airways and HTII-280⁺ alveolar type II progenitors in the distal airways. scale bare is 50 μm.

FIG. 2 (panels A-H) depicts an exemplary sorting strategy for distal lung (A & E) Percentage of various cell populations before and after depletion of CD45⁺ and CD31⁺ population using CD31 and CD45 magnetic beads in distal regions of the lung. (B & F) Representative image of FACS plot showing gating strategy of distal CD31⁻/CD45⁻/CD235a⁻ population before and after depletion of CD31/CD45/CD235a positive population (C & F) Epcam⁺ population before and after depletion of CD31/CD45/CD235a positive population. (D & H) HT II-280^{+/-} population before and after depletion of CD31/CD45/CD235a positive cells. FIGS. 2A, 2B, 2C and 2D are from the same biological sample and FIGS. 2E, 2F, 2G and 2H are from the same biological sample.

FIG. 3 (panels A-H) depicts an exemplary sorting strategy for proximal lung (A & E) Percentage of various cell populations before and after depletion of CD45+ and CD31+ population using CD31 and CD45 magnetic beads in proximal regions of the lung. (B & F) Representative image of FACS plot showing gating strategy of proximal CD31⁻/CD45⁻/CD235a⁻ population before and after depletion of CD31/CD45/CD235a positive population (C & F) Epcam⁺ population before and after depletion of CD31/CD45/CD235a positive population. (D & H) NGFR^{+/-} population before and after depletion of CD31/CD45/CD235a positive cells. FIGS. 3A, 3B, 3C and 3D are from the same biological sample and FIGS. 3E, 3F, 3G and 3H are from the same biological sample.

FIG. 4 (panels A-D, C′ D′) depicts characterization of distal organoids (A, B) Representative image of the human distal organoids cultured in PneumaCult™-ALI medium (2×) and (10×). (B) The Colony forming efficiency (% CFE) was calculated on triplicate wells of organoids derived from two biological samples. (C, D) Immunofluorescent staining of corresponding distal organoid cultures in ALI medium showing HT II-280⁺ AT2 cells (green) and nuclei (blue). (C′, D′) higher magnification of Immunofluorescent staining of corresponding distal organoids cultured in PneumaCult™-ALI medium showing HTII-280+AT2 cells (green) and nuclei (scale bar=50 mm).

FIG. 5 (panels A-F) depicts characterization of Proximal organoids from the human Proximal lung (A, B) Representative image of the human Proximal organoids cultured in PneumaCult™-ALI medium scale bar 50 mm. (C) The Colony forming efficiency (% CFE) was calculated on triplicate wells of organoids derived from three biological replicates. (D) Immunofluorescent staining of differentiated proximal organoids expressing Acetylated tubulin (red). (E) FOXJ1 (red) marking ciliated cells (F) goblet cells expressing MUC5AC (red). (E, F) basal cells expressing K5 (green) at day 30. scale bar=50 mm. Table 1: Cell isolation

FIG. 6 (panels A-I) shows comparing sorting strategy for mouse lung (Fresh Tissue VS Frozen Tissue Vs frozen Lung cells): A, B & C are from same biological sample (Fresh mouse lung). (A) Flow cytometry of mouse fresh lung cells suspension showing gating strategy. (B) Representative image of FACS plot showing gating strategy of CD31−/CD45-population and (C) Epcam+ population. D, E & F are from same biological sample (Frozen mouse lung tissue). (D) Flow cytometry of cells isolated from mouse frozen lung tissue, showing gating strategy. (E) FACS plot showing gating strategy of CD31−/CD45-population and (F) Epcam+ population. G, H & I are from same biological sample (frozen mouse lung cells). (G) Flow cytometry of frozen mouse lung cells showing gating strategy. (H) FACS plot showing gating strategy of CD31−/CD45 population and (I) Epcam+ population.

FIG. 7 (Panels A-L) shows comparing sorting strategy for human distal lung (Fresh Tissue VS Frozen Tissue Vs Frozen Lung cells): A to L are from same biological sample with different conditions (Fresh Tissue, Frozen Tissue and Frozen cells) and sorted on same day. (A) gating strategy for cells isolated from fresh distal lung tissue, (B) percentage of live cells by DAPI staining, (C) gating strategy for CD31⁻/CD45⁻ population, (D) Epcam⁺ and HT II-280⁺ population. (E) showing gating strategy for cells isolated from frozen distal lung tissue, (F) percentage of live cells by DAPI staining, (G) gating strategy for CD31⁻/CD45⁻ population and (H) Epcam⁺ and HT II-280 k population. (I) showing gating strategy for frozen distal lung cells, (J) percentage of live cells by DAPI staining, (k) gating strategy for CD31⁻/CD45⁻ population and (L) Epcam⁺ and HT II-280⁺ population.

FIG. 8 (panels A-G) depict Colony Forming Efficiency of HT II-280⁺ cells from Human Distal lung. Fresh Lung Tissue Vs Frozen Lung Tissue Vs Frozen Cells. A, B, C & D are from same biological sample. (A) Representative images of organoids formed from HT II-280⁺ cells, day 20 from Fresh distal lung, (B) Frozen Distal tissue and (C) Frozen distal lung cells. 2000 cells/well were added and cultured in SAEGM medium. (D) Colony Forming Efficiency of HT II-280⁺ cells from the corresponding sample measured on day 20. Representative images of organoids formed from HT II-280⁺ cells from two different biological distal lung samples. 5000 cells/well were cultured in PneumaCult™ ALI medium for 30 days. (E) Organoids culture from Fresh distal lung tissue, (F) Organoids cultured from Frozen Tissue. (G) Colony Forming Efficiency of HT 11-280+ cells on day 30 from two biological samples with triplicates.

FIG. 9 (panels A-L) shows Comparing Sorting Strategy for Human Proximal Lung (Fresh VS Frozen Vs Frozen Lung cells): A to L are from same biological sample with different conditions (Fresh Tissue, Frozen Tissue and Frozen cells) and sorted on same day. (A) gating strategy for cells isolated from fresh proximal lung tissue, (B) gating strategy for CD31⁻/CD45⁻ population, (C) Epcam⁺ and (D) NGFR⁺ population. (E) showing gating strategy for cells isolated from frozen proximal lung tissue (F) gating strategy for CD31⁻/CD45⁻ population, (G) Epcam⁺ and (H) NGFR⁺ population. (I) showing gating strategy for proximal frozen lung cells, (J) gating strategy for CD31⁻/CD45⁻ population, (K) Epcam⁺ and (L) NGFR⁺ population.

FIG. 10 (panels A-G) depicts Colony Forming Efficiency of NGFR⁺ cells from Human Proximal lung. Fresh Lung Tissue Vs Frozen Lung Tissue Vs Frozen Cells. A, B, C & D are from same biological sample. (A) Representative images of organoids formed from NGFR⁺ cells, day 20 from Fresh proximal lung, (B) Frozen proximal tissue and (C) Frozen proximal lung cells. 2000 cells/well were added and cultured in PneumaCult EX Basal medium. (D) Colony Forming Efficiency of Epcam⁺ cells from the corresponding sample measured on day 20. (E, F & G) Representative images of organoids formed from NGFR⁺ cells from two different biological Proximal lung samples. 5000 cells/well were cultured in PneumaCult™ ALI medium for 30 days. (E) Organoids culture from Fresh proximal lung tissue, (F) Organoids cultured from Frozen Tissue. (G) Colony Forming Efficiency of NGFR⁺ cells on day 30 from two biological samples with triplicates.

FIG. 11 (panels A-G) shows Comparing Colony Forming Efficiency and Sorting profile in Fresh tissue vs Frozen tissue for both Distal Lung and Proximal Lung. A to H are from same biological sample. (A & B) Organoids formed from HT II-280⁺ cell and FACS profile of Human Fresh Distal tissue. (C & D) Organoids formed from HT II-280⁺ cell and FACS profile of Frozen Distal tissue. (E & F) organoids formed from NGFR⁺ cell and FACS profile of Fresh Proximal tissue. (G & H) Organoids formed from NGFR⁺ cell and FACS profile of Frozen Proximal tissue (A & C) Colony Forming Efficiency is similar in both fresh and frozen distal tissue and (E & G) Proximal fresh and frozen tissue.

FIG. 12 (panels A-B) depicts Characterization and Comparing Immunohistochemistry of Distal Organoids from Fresh Tissue Vs Frozen Tissue. (A) Representative immunofluorescent staining of human fresh distal organoids cultured in PneumaCult™-ALI and (B) frozen distal organoids showing HT II-280⁺ AT2 cells (green) and nuclei (blue). (scale bar=50 mm).

FIG. 13 (panels A-C) depicts Characterization and Comparing Immunohistochemistry of Proximal Organoids from Fresh Tissue Vs Frozen Tissue. (A) Immunofluorescent staining of differentiated proximal organoids expressing MUC5AC (red) in goblet cells and basal cells expressing K5 (green) at day 30 from fresh Proximal lung tissue and (B) Frozen proximal lung tissue. (C) FOXJ1 (red) marking ciliated cells and basal cells expressing K5 (green) at day 30 from fresh proximal tissue. (scale bar=50 mm).

FIG. 14 shows a schematic representation of Isolation of single cells from human and mouse lung. Experimental workflow for extracting of epithelial progenitor cells from human lungs and mouse lungs. Dissociate the lung using enzymes such as liberase, dispase and elastase. Isolating single cells and lysing Red Blood cells by adding RBC lysis buffer. Performed optional depletions step for depleting CD45⁺ cells and CD31⁺ cells using magnetic beads which enhance sorting efficiency and saves sorting time. Cell surface staining performed to sort cells using Influx. Collecting HT II 280⁺ cells after sorting and coculture with MRCS for organoid culture.

FIG. 15 (panels A-E) shows a representative sorting Strategy for Fresh tissue, frozen tissue and frozen cells from the mouse lung. Representative flow cytometry manual gating strategy of live single cells; Image showing the comparison between FACS plots of CD31⁻/CD45⁻/CD326⁺ population from fresh tissue (A), frozen tissue (B) and frozen cells (C). This experiment is performed on n=9 animals with 3 animals per group. Sorting is performed on same day for all the 9 animals. Based on the profile, there is no significant p value difference in cell viability between fresh and frozen mouse lung tissue and its slightly lower in frozen mouse lung cells (D). 5000 Epcam⁺ cells are co-cultures with MLG's for organoid culture. Colony forming Efficiency was measure on Day 7 and it's similar between fresh and frozen mouse lung tissue with no significant P value (E).

FIG. 16 (panels A-E) shows representative sorting Strategy for Fresh tissue, frozen tissue and frozen cells for distal human lung Comparing Sorting Strategy for Human Distal Lung. Representative image showing the comparison between FACS plots of CD31⁻/CD45⁻/CD326⁺/HTII-280⁺ population from fresh distal lung tissue (A), frozen lung distal tissue pieces (B) and frozen dissociated human distal lung cells (C). Based on FACS profile % of Epcam⁺ and % of HT II 280⁺ are almost similar in all the groups. Immunofluorescent staining of organoid cultures from corresponding fresh lung tissue (A) and frozen lung tissue (B) and frozen distal lung cells (C) in Pneumacult ALI medium at day 25 showing HT II-280⁺ AT2 cells (green) and nuclei (blue). Scale bar=50% of cell viability in single cells isolated from distal lung compared between fresh and frozen lung in 11 different biological sample (D). No significant P value between the fresh and frozen tissue groups. Organoids are cultured from HT II 280⁺ cells and colony forming efficiency was calculated on triplicate wells of organoids derived from different biological sample with seeding density of 5000 cells/well on day 15(E).

FIG. 17 (panels A-H) show 10× data comparing between fresh and frozen distal lung tissue. (A) Evaluation of number of transcripts, Unique Molecular Identifiers (HMI), and percentage of mitochondrial genes, in datasets derived from fresh and frozen tissue, visualized by violin plots. Red arrows indicate abundance of low reads for transcripts and UMI in the ‘frozen’ dataset. (B) Dimensional reduction of data generated from freshly isolated Fresh and Frozen tissue, visualized by UMAP, with cells colored by subset as shown in key. (C-H) Expression of cell-type specific transcripts, divided by ‘fresh’ and ‘frozen’ datasets, visualized by UMAP.

FIG. 18 (panels A-C) shows representative sorting strategy for mouse lung cells and organoids.

FIG. 19 (panels A-C) shows representative Sorting Strategy for Human Distal Lung Cells and Characterization of Distal Lung Organoids.

FIG. 20 depicts sorted that cells were stained for HTII 280 and SPC.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

One skilled in the art will recognize many methods and materials similar or equivalent to those described herein, which could be used in the practice of the present invention. Indeed, the present invention is in no way limited to the methods and materials described.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Modulation” or “modulates” or “modulating” as used herein refers to upregulation (i.e., activation or stimulation), down regulation (i.e., inhibition or suppression) of a response or the two in combination or apart.

“Pharmaceutically acceptable carriers” as used herein refer to conventional pharmaceutically acceptable carriers useful in this invention.

“Promote” and/or “promoting” as used herein refer to an augmentation in a particular behavior of a cell or organism.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Proximal” region of a lung as used herein refers to the region of the lung from the trachea along with the bronchi up to the first branching.

“Distal” region of a lung as used herein refers to small airways (less than 2 mm in diameter along with the surrounding tissue.

This protocol generated robust yields of region-specific epithelial progenitor cells from frozen distal lung tissue. The isolated cells were analyzed using single cell RNA sequencing and in vitro organoid assays, which provide insights into the molecular and functional capacities of these cells. Together, these techniques can also help resolve subpopulations of epithelial cells, and enable us to refine our understanding of unique cellular processes exhibited in the distal lung microenvironment.

Advantages of cryobanked human lung tissue include but are not limited to: (1) Frozen lung tissue provides better logistical flexibility, as samples can be shipped in a manner similar to other frozen cells for analysis at different sites; (2) Lung samples collected from different patients at different times can be cryobanked for simultaneous processing for either genomic or functional analysis; (3) Viability of cells isolated following application of this cryobanking protocol is comparable to that seen with freshly collected tissue; and (4) Cryopreservation can be done not only in human samples but also can be done with mouse and ferret lung.

Herein, we describe a reliable method for freezing human lung tissue, which can be used for the subsequent isolation of region-specific cell populations for downstream analysis. Critical elements of this method include preserving the lung tissue, increasing cellular viability, and enhancing recovery of these cells from frozen tissues followed by large-scale expansion of organoids in vitro.

By culturing organoids derived from frozen tissues and comparing the resulting colony forming efficiency of both organoids from fresh tissue and frozen cells, we can evaluate the efficacy of our protocol alongside other methods.

Freezing lung tissue using this optimized protocol not only allows for downstream applications with results consistent with freshly procured tissue, but also provides practical advantages that may benefit any research program. As specific lung samples—such as normal lungs, diseased lungs, or lungs of a particular genetic background—may not always be readily available, having the ability to maintain frozen stocks of reliable samples serves to mitigate the challenges presented by the reliance on external sources of biological samples. Furthermore, this platform enables on-demand experimentation, and can facilitate collaborative efforts by easing the tissue-sharing process among local or distant investigators.

Previously, after receiving human lung tissue weighing approximately 1000 grams, processing of the entire sample must be performed the same day. Significant amount of lab resources, including personnel, reagents, and time, were required for the isolation and banking of cells. A significant benefit that this novel protocol provides is that the isolation of dissociated cells need not proceed immediately. Rather, one investigator may freeze the lung sample according to this protocol, allowing for further processing of the tissue in the future, on a smaller-scale, according to the experimental need.

Current single cell RNA seq protocols require samples to be processed on the same day as procurement to prevent changes to transcriptomic signature of the cells. However, single-cell RNAseq analysis of human lung tissue cryopreserved using our method preserved the cellular heterogeneity and gene expression signatures that were seen in freshly isolated specimens. This is a significant practical advancement as it will allow for a way to store normal and diseased lung tissue based on availability and process samples for single cell RNA seq without compromising their transcriptomic signatures. Our cryopreservation method also represents a relevant approach to minimize batch effects driven by processing related samples at different times.

Using this protocol, we can preserve not only distal lung from healthy donors, but also tissue obtained from patients with Idiopathic Pulmonary Fibrosis, Cystic Fibrosis, COPD, and others. We can isolate cells from frozen IPF lung and perform 10× single cell RNA seq, which reveals profibrotic roles of distinct epithelial and mesenchymal lineages and also helps in identifying patterns of intracellular heterogeneity.

Various embodiments of the present invention are based, at least in part, upon the finding discussed herein.

Various embodiments of the present invention provide for a method of processing and optionally freezing lung tissues, cells or both, comprising: cutting lung tissue into about 0.5 cm³ to 2.0 cm³ pieces; washing the pieces of tissue to remove blood, epithelial lining fluid, or both; optionally drying the pieces of lung tissue; removing visceral pleura from the pieces of lung tissue; and further cutting the pieces of lung tissue into about 0.5-5.0 mm diameter pieces, wherein the lung tissue is the proximal region of the lung or the distal region of the lung.

In various embodiments, the method further comprises identifying and separating the proximal and distal regions before cutting the tissue into about 0.5 cm³ to 2.0 cm³ pieces.

In various embodiments, the method comprises freezing the lung tissues, and the method further comprises: placing the about 0.5-5.0 mm diameter pieces of lung tissue into a vial and cryoprotective media; and freezing the vials comprising the 0.5-5.0 mm diameter pieces of lung tissue and cryoprotective media to a temperature of about −90 to −70 degrees C. In various embodiments, freezing the vials to a temperature of about −90 to −70 degrees C. comprises freezing the vials to about −80 degrees C. In various embodiments, this freezing is performed separately and before freezing the pieces of lung tissue in a liquid nitrogen, or in vapor phase of liquid nitrogen. In various embodiments, the method further comprises freezing the pieces of lung tissue in vapor phase of a liquid nitrogen vessel. For example, the vial containing the pieces of lung tissue is frozen in vapor phase of a liquid nitrogen vessel. “Vial” as used herein refers to any container suitable for storing tissues or cells; particularly, storing tissues or cells at the temperatures indicated herein.

In various embodiments, the about 0.5-5.0 mm diameter pieces placed into the vial and cryoprotective media are about 2-5 mm diameter pieces, or about 3-4 mm diameter pieces. In various embodiments, the about 0.5-5.0 mm diameter pieces placed into the vial and cryoprotective media are about 0.5, 1.0, 1.5, 2.0, 2.5, 3.0, 3.5, 4.0, 4.5, or 5.0 mm diameter pieces.

In various embodiments, cutting lung tissue into about 0.5 cm³ to 2.0 cm³ pieces comprising cutting lung tissue into about 1.0 cm³ pieces. In various embodiments, the tissues are cut into about 0.5, 0.75, 1.0, 1.25, 1.5, 1.75 or 2.0 cm³ pieces.

In various embodiments, further cutting the lung tissue into about 0.5-5.0 mm diameter pieces comprising cutting the lung tissue into about 2-5 mm diameter pieces. In various embodiments, further cutting the lung tissue into about 0.5-5.0 mm diameter pieces comprising cutting the lung tissue into about 3-4 mm diameter pieces. In various embodiments the tissue is cut into about 0.5, 1.0, 1.5, 2.0, 2.5, 3.0, 3.5, 4.0, 4.5, or 5.0 mm diameter pieces.

Various embodiments of the present invention provide for a method of enrichment and optionally sub setting of small airway and aveolar epithelial progenitor cells from distal lung tissue, comprising: performing the method of processing and optionally freezing lung tissues, cells or both as described herein, wherein the lung tissue is distal lung tissue, and further cutting the lung tissue into about 0.5-5.0 mm diameter pieces comprises cutting the lung tissue into about 0.5-1.5 mm diameter pieces; and digesting the about 0.5-1.5 mm diameter pieces of lung tissue with enzyme; dissociating the digested pieces of lung tissue into single cells; and selecting epithelial progenitor cells.

In various embodiments, cutting the lung tissue into about 0.5-1.5 mm diameter pieces comprises first cutting the 0.5-5.0 mm diameter pieces of lung tissue into about 1.5-2.5 mm diameter pieces, and then cutting the 1.5-2.5 mm diameter pieces into about 0.5-1.5 mm diameter pieces. In various embodiments, cutting the lung tissue into about 0.5-1.5 mm diameter pieces comprise cutting the lung tissue into about 0.5, 0.75, 1.0, 1.25, or 1.5 mm diameter pieces. In various embodiments, cutting the 1.5-2.5 mm diameter pieces into about 0.5-1.5 mm diameter pieces comprise cutting the 1.5-2.5 mm diameter pieces into about 1.0 mm diameter pieces.

In various embodiments, the enzyme comprises collagenase I, collagenase II, a non-clostridial neutral protease, or DNase, or combinations thereof.

In various embodiments, selecting the epithelial progenitor cells comprising selecting cells having a surface marker profile that is one or more of: CD45-negative, CD31-negative, and CD236-positive, and optionally have a negative staining for DAPI.

In various embodiments, the method further comprises sub setting of small airway and aveolar epithelial progenitor cells, the method comprising: selecting epithelial cells that are HTII-280-negative as small airway epithelial progenitor cells; or selecting epithelial cells are HTII-280-positive as alveolar type 2 (AT2) progenitor cells.

In various embodiments, selecting epithelial progenitor cells comprising depleting immune cells and endothelial cells, cell surface staining for fluorescence associated cell sorting (FACS), or both.

Various embodiments provide for a method of enrichment and optionally subsetting of epithelial progenitor cells from trachea-bronchial airways, comprising: performing the method of processing and optionally freezing lung tissues, cells or both as described herein, wherein the lung tissue is the proximal region of the lung, and wherein luminal epithelial cells have been removed from the proximal region of the lung; digesting the pieces of lung tissue with enzyme; dissociating the digested pieces of lung tissue into single cells; and selecting epithelial progenitor cells.

In various embodiments, the method further comprises performing the following steps before performing the method of processing and optionally freezing lung tissues, cells or both as described herein: opening airways of the distal lung tissue along their length to expose their lumen and cover the tissue with a solution comprising collagenase I, collagenase II, a non-clostridial neutral protease, or a combination thereof; stripping the luminal epithelial cells from the tissue; and collecting the luminal epithelial cells.

In various embodiments, the method further comprises dissociating luminal epithelial cells into single cells.

In various embodiments, method further comprises combining the single cell luminal epithelial cells with the single cells from the proximal region before selecting for epithelial progenitor cells.

In various embodiments, the enzyme comprises collagenase I, collagenase II, a non-clostridial neutral protease, or DNase, or combinations thereof.

In various embodiments, selecting the epithelial progenitor cells comprising selecting cells having a surface marker profile that is one or more of: CD45-negative, CD31-negative, and CD236-positive, and optionally have a negative staining for DAPI.

In various embodiments, the method further comprises sub setting of epithelial progenitor cells, the method comprising: selecting epithelial progenitor cells that are NGRF-positive as a basal cell type; or selecting epithelial progenitor cells that are NGRF-negative as a non-basal cell type.

In various embodiments, selecting epithelial progenitor cells comprising depleting immune cells and endothelial cells, cell surface staining for fluorescence associated cell sorting (FACS), or both.

In various embodiments, the lung tissue used in these methods is fibrotic lung tissue. In various embodiments, the fibrotic lung tissue is from a subject with idiopathic lung fibrosis. In various embodiments, the fibrotic lung tissue is idiopathic lung fibrotic tissue.

Various embodiments of the present invention provide for a method of generating a lung organoid, comprising: providing a quantity of cells of made by or isolated by any one of the methods described herein; culturing the cells in the presence of a growth media comprising a Rho kinase inhibitor; and further culturing the cells in the presence of a TGFβ inhibitor to generate lung organoids.

In various embodiments, the Rho kinase inhibitor is Y-27632. In various embodiments, the TGFβ inhibitor is SB431542. In various embodiments, the fluidic device is a transwell system. In various embodiments, culturing the cells is for a period of about 7-40 days. In various embodiments, the method further comprise culturing the cells is for a period of about 15 days. Various embodiments provide for a quantity of lung organoids made by a method described herein.

Various embodiments of the present invention provide for a system for modeling a lung, comprising: a population of cells comprising cells selected from the group consisting of lung cells isolated by any one of the methods described herein, primary lung cells differentiated from lung cells isolated by any one of the methods described herein, a lung organoid comprising the lung cells or the primary lung cells; and a cell culture device, a cell culture plate, or a multi-well culture plate.

In various embodiments, the population of cells are in a cell culture device, a cell culture plate, or a multi-well culture plate.

Various embodiments provide for a system for test agent screening in a lung model, comprising: a population of cells comprising cells selected from the group consisting of lung cells isolated by any one of the methods as described herein, primary lung cells differentiated from lung cells isolated by any one of the methods described herein, a lung organoid comprising the lung cells or the primary lung cells; a cell culture device, a cell culture plate, or a multi-well culture plate; wherein the test agent and the population of cells, are in contact in the cell culture plate, or the multi-well culture plate.

In various embodiments, the cell culture device is an air-liquid interface culture or a Transwell system comprising the population of cells.

In various embodiments, the lung cells are epithelial cells. In various embodiments, the epithelial cells are small airway epithelial progenitor cells, alveolar type 2 (AT2) progenitor cells, basal cell type, or non-basal cell type. In various embodiments, the epithelial cells are proximal airway cells, or distal alveolar cells.

In various embodiments, the lung model is a lung fibrosis model. In various embodiments, the lung fibrosis is idiopathic lung fibrosis.

Various embodiments provide for a method selecting an agent of interest, comprising: contacting a test agent with a population of cells comprising cells selected from the group consisting of lung cells isolated by any one of the methods described herein, primary lung cells differentiated from lung cells isolated by any one of the methods of described herein, a lung organoid comprising the lung cells or the primary lung cells, wherein the test agent and the population of cells are in contact in a cell culture device, a cell culture plate, or a multi-well culture plate; measuring a parameter in the population of cells; and selecting the test agent as the agent of interest based on the measured parameter in the population of cells.

In various embodiments, the parameter comprises a phenotype of interest, an expression level of a gene, or an expression level of a protein of interest, or combination thereof.

In various embodiments, cell culture device is an air-liquid interface culture or a Transwell system comprising the population of cells.

In various embodiments, the lung cells are epithelial cells. In various embodiments, the epithelial cells are small airway epithelial progenitor cells, alveolar type 2 (AT2) progenitor cells, basal cell type, or non-basal cell type. In various embodiments, the epithelial cells are proximal airway cells, or distal alveolar cells.

In various embodiments, the lung cells are isolated from fibrotic lung tissue. In various embodiments, the lung cells are isolated from idiopathic lung fibrotic tissue.

Various embodiments provide for a method modeling a lung, comprising: measuring a parameter in a population of cells comprising cells selected from the group consisting of lung cells isolated by any one of the methods described herein, primary lung cells differentiated from lung cells isolated by any one of the methods described herein, a lung organoid comprising the lung cells or the primary lung cells, wherein the population of cells are in contact in a cell culture device, a cell culture plate, or a multi-well culture plate.

In various embodiments, the method further comprises contacting a test agent to the population of cells before, while, after or a combination thereof, measuring the parameter. In various embodiments, the method further comprises contacting a test agent to the population of cells before measuring the parameter. In various embodiments, the method further comprises contacting a test agent to the population of cells while measuring the parameter. In various embodiments, the method further comprises contacting a test agent to the population of cells after measuring the parameter.

In various embodiments, the parameter comprises a phenotype of interest, an expression level of a gene, or an expression level of a protein of interest, or combination thereof.

In various embodiments, the cell culture device is an air-liquid interface culture or a Transwell system comprising the population of cells.

In various embodiments, the lung cells are epithelial cells. In various embodiments, the epithelial cells are small airway epithelial progenitor cells, alveolar type 2 (AT2) progenitor cells, basal cell type, or non-basal cell type. In various embodiments, the epithelial cells are proximal airway cells, or distal alveolar cells.

In various embodiments, the lung cells are fibrotic lung cells. In various embodiments, the lung cells are idiopathic fibrotic lung cells.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Differentiation Protocol

Human lung tissue was obtained from deceased tissue donors in compliance with consent procedures developed by International Institute for the Advancement of Medicine (IIAM) and approved by the Cedars-Sinai Medical Center Internal Review Board.

Tissue Processing for Isolation of Lung Cells from Either Tracheo-Bronchial or Small Airway/Parenchymal (Small Airways and Alveoli) Regions:

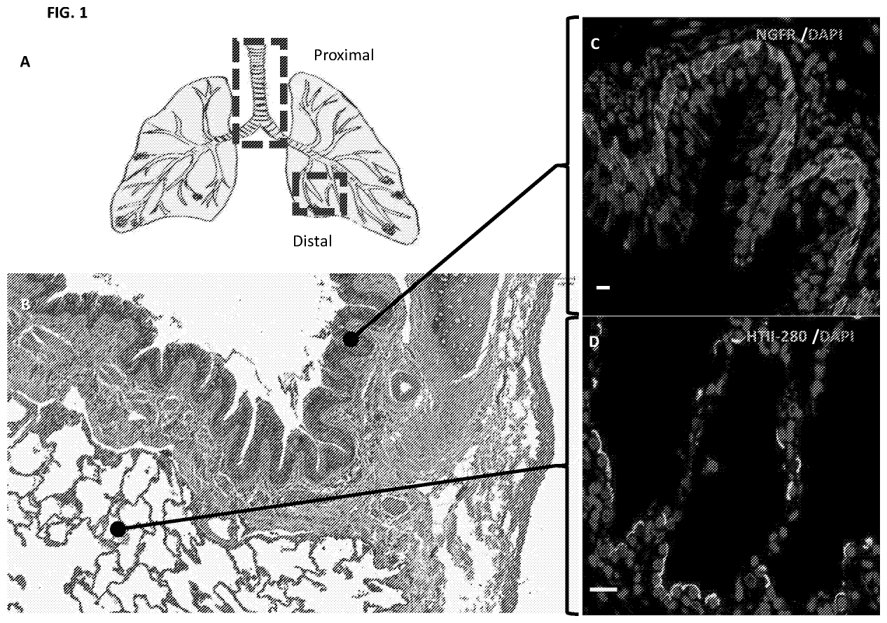

Prepare and autoclave all dissection instruments, glassware and the appropriate solutions one day prior to cell isolation. Upon receiving lung tissue identify and separate the proximal and distal regions as shown in the video. Briefly, the trachea along with the bronchi up to the first branching was considered ‘proximal’ and small airways (less than 2 mm in diameter) along with the surrounding tissue were considered ‘distal’ (FIG. 1A)

Enrichment and Sub Setting of Small Airway and Alveolar Epithelial Progenitor Cells from Distal Lung Tissue:

### Distal Tissue Preparation:

Place distal lung tissue in a sterile petri dish (150×15 mm) in a biosafety cabinet. Dice tissue into approximately 1 cm³ pieces and place in a clean 50 mL tube. Wash tissue three times with chilled HBSS, discarding the HBSS wash each time to remove blood and epithelial lining fluid.

Place tissue in a new petri dish and blot dry with sterile Kim wipes. Using forceps and scissors, remove as much visceral pleura as possible. Use scissors to mince tissue into pieces of approximately 2 mm diameter.

Transfer minced tissue into a clean petri dish and mince further by chopping to an approximate size of 1 mm with a sterile single sided razor blade.

### Enzyme Digestion:

Note: Liberase™ stock solution is 5 mg/mL (100×) and DNase stock is 2.5 mg/mL (100×). (Liberase™ contains highly purified Collagenase I and Collagenase II. These two collagenase isoforms are blended in a precise ratio to each other, and with a medium concentration of Thermolysin, a non-clostridial neutral protease.)

Add 50 μg/mL Liberase™ and 25 μg/mL DNase solution solutions to sterile HBSS in a 50 mL conical tube.

Transfer minced tissue to a 50 mL conical tube and incubate for 40-60 minutes at 37° C. with continuous shaking using a Thermomixer set at 900 rpm.

Note: Incubation time in the enzymes can vary depending on the type or condition of the tissue. For example, enzymatic digestion of normal tissue takes approximately 45 minutes. However fibrotic tissue from IPF samples can require a longer incubation time of up to 60 minutes. Therefore, tissue should be carefully monitored during this step to prevent damage to the surface markers which is crucial for FACS and incubation times should be standardized based on the tissue type.

### Single Cell Isolation:

Triturate tissue by drawing 5 times through a 16 G needle fitted to a 60 mL syringe. Draw tissue suspension into a wide-bore pipette and pass through a series of PluriSelect cell strainers (500 μm, 300 μm, 100 μm, 70 μm, 40 μm) under vacuum pressure. Wash the strainer with 20 mL of HBSS+ buffer to collect remaining cells. The recipe for HBSS+ buffer can be found in Table 2.

Add an equal volume of HBSS+ buffer to the filtrate to inhibit Liberase activity and prevent over-digestion.

Centrifuge filtrates at 500×g for 5 minutes at 4° C.

Carefully remove and discard the supernatant. Add 1 mL of Red Blood Cell (RBC) lysis buffer to the pellet, gently rock the tube to dislodge the pellet and incubate on ice for 1 minute.

Note: The amount and time in the RBC lysis solution depends upon the size of the pellet. It is important to maintain the cells on ice and monitor time in RBC lysis solution carefully to prevent lysis of target cells. If RBC lysis is insufficient, the Inventors repeat the step.

Add 10-20 mL HBSS+ buffer to neutralize RBC lysis buffer. Centrifuge filtrates at 500×g for 5 minutes at 4° C.

Note: Lysed red blood cells (ghost cells) may form a cloudy layer above the cell pellet. In this case, resuspend pellet in 10 mL of HBSS+ buffer and strain the suspension through 70 μm cell strainer to eliminate the ghost cells. Centrifuge filtrate at 600×g for 5 minutes at 4° C. and proceed with further steps.

### Depletion of Immune Cells, Endothelial Cell (Optional Step):

Deplete CD31⁺ endothelial cells and CD45⁺ immune cells from the pool of total cells using the Milteny MACS CD31 & CD45 microbeads conjugated to monoclonal anti-human CD31 and CD45 antibody (isotype mouse IgG1) and LS columns in accordance to the manufacturer's protocol.

Collect flow through consisting primarily of epithelial and stromal cells in a fresh sterile tube and centrifuge it at 600×g for 5 mins at 4° C. Perform a cell count to ascertain the total number of cells in the flow through.

### Cell Surface Staining for Fluorescence Associated Cell Sorting (FACS):

Resuspend 1×10⁷ cells per 1 mL of HBSS+ buffer. Add primary antibodies at the required concentration and incubate the cells for 30 mins at 4° C. in dark. In this study, fluorophore conjugated primary antibodies were used unless otherwise stated. Details of antibody sources and titers are described in Table 5.

Note: HTII-280 is currently the best surface reactive Ab that allows subsetting of distal lung cells into predominantly airway (HTII-280⁻) and alveolar (HTII-280⁺) fractions. A caveat to this strategy is that AT1 cells are not stained using this method. However, AT1 cells are poorly represented in distal lung preps, presumably due to their fragility and loss during selection of viable cells by FACS and thus only represent a rare contaminant of the airway cell fraction).

Wash cells by adding 3 mL of HBSS+ buffer and centrifuge at 600×g for 5 minutes at 4° C.

If using unconjugated primary antibodies, add required concentration of an appropriate fluorophore conjugated secondary antibody and incubate for 30 mins on ice.

Wash off excess secondary antibody by adding 3 mL of HBSS+ buffer and centrifuge at 600×g for 5 mins at 4° C.

Discard supernatant and resuspend cells in HBSS+ buffer per 1×10⁷ cells/mL.

Filter cells into 5 mL polystyrene tubes through a strainer cap to ensure formation of a single cell suspension.

Add DAPI (1 μg/mL) to stain permeable (dead) cells.

Note: It is essential to use appropriate single-color and Fluorescence minus one (FMO) controls (i.e. antibody staining cocktail minus one antibody each), to minimize false positives during FACS. In this study the Inventors use positive and negative selection beads for empirical compensation for overlap of emission spectra between fluorophores (Table 5).

Note: FACS enrich cell types of interest. Viable epithelial cells are enriched based upon their CD45-negative, CD31-negative, CD236-positive cell surface phenotype and negative staining for DAPI. This epithelial cell fraction can be further subsetted based on staining for cell type-specific surface markers, such as specific staining for HTII-280-positive cells that are enriched for AT2 cells. In contrast, negative selection for HTII-280 allows the enrichment of small airway epithelial cells such as club and ciliated cells.

Enrichment and Subsetting of Epithelial Progenitor Cells from Tracheo-Bronchial Airways:

### Tissue Preparation:

Dissect out proximal airways (trachea/bronchi) from the lungs. Open airways along their length using scissors to expose the lumen and add 50 μg/mL Liberase™ to fully cover the tissue. Incubate for 20 minutes at 37° C. with continuous shaking using a Thermomixer set at 900 rpm.

Remove proximal airway from centrifuging tube and place it in a sterile petri dish (150×15 mm). Gently scrape the surface of the airway using a scalpel to completely strip luminal epithelial cells from the tissue. Wash the petri dish with a sterile 5 mL of HBSS+ buffer to collect all dislodged luminal epithelial cells and transfer the dislodged cells to 50 mL Falcon tube.

Triturate suspension by drawing 5 times through 16 G needle and 18 G needle fitted to a 10 mL syringe to get single cell suspension.

Centrifuge suspension at 500×g for 5 mins at 4° C. Resuspend the pellet in a fresh HBSS+ buffer and store these luminal airway cells on ice, ready to be combined with the single cell suspension generated from the minced proximal airways in the further steps.

Using scissors, cut remaining tracheobronchial tissue along its rings to generate small strips of tissue and transfer the strips to a fresh petri dish.

Mince the tissue strips using a single sided razor blade to make smaller pieces. NOTE: Since the proximal airways are cartilaginous, they cannot be minced as finely as finely as the distal lung tissue.

Transfer minced tissue into the Gentle MACS C tubes, add 2 mL of Liberase™ to the tube ensuring that the tissue is submerged.

Load the C tube onto the Gentle MACS Octo Dissociator (Table 3) and run Human Lung Protocol-2 to mechanically dissociate tissue further.

Note: MACS Octo Dissociator offers an optimized gentle MACS program called human lung protocol-2 for this specific application.

### Enzyme Digestion and Single Cell Isolation:

Transfer approximately 2 gm of minced proximal tissue from the C tube into each 50 mL Falcon tube and add 50 μg/mL Liberase™ and 25 μg/mL DNase solution to each tube.

Note: To ensure efficient dissociation, tubes should not be filled beyond the 30 mL mark.

Incubate the minced tissue for 45 minutes at 37° C. with continuous shaking using a Thermomixer set at 900 rpm

Pass the dissociated tissue suspension through a series of PluriSelect cell strainers (500 μm, 300 μm, 100 μm, 70 μm, 40 μm) under vacuum pressure as mentioned above and collect the flow through. Wash the strainer with 20 mL of HBSS+ buffer to collect remaining cells.

Note: Since proximal tissue is cartilaginous and bulky as compared to the distal tissue, there is a higher possibility of clogging of the filters. Using a funnel as shown in the video can help prevent overflowing of the liquid while passing through the strainers.

Add an equal volume of HBSS+ buffer to the filtrate to inhibit Liberase activity and prevent over-digestion.

Add the isolated luminal proximal airway cells from 3.1.4 to the cell suspension at this step.

Centrifuge the combined cell suspension at 600×g for 10 mins. Remove supernatant and repeat cell wash in HBSS+ buffer.

Perform depletion of CD45⁺ immune cells and CD31⁺ endothelial cells as mentioned above in 2.4

### Cell Surface Staining for Flow Cytometry:

Methods for staining proximal airway cells are as described for distal lung tissue. Viable epithelial cells are enriched based upon their CD45-negative, CD31-negative, CD236-positive cell surface phenotype and negative staining for DAPI. This epithelial cell fraction can be further subsetted based upon staining for cell type-specific surface markers, such as NGFR, allowing enrichment of basal (NGFR-positive) and non-basal (NGFR-negative; secretory, ciliated, neuroendocrine) cell types.

### Organoid Culture (Table 5):

Add 2000-5000 sorted proximal or distal epithelial cells and 7.5×10⁴ MRCS cells to a sterile 1.5 mL tube.

Centrifuge at 500×g for 5 mins at 4° C.

Note: It is important to manually confirm the cell count obtained from the sorter in order to ensure accuracy organoid colony forming efficiency.

Carefully remove and discard the supernatant and resuspend the cell pellet in 50 μl of ice-cold media supplemented with antibiotics. Keep the cell suspension on ice.

Add 50 μl of ice cold 1× growth factor depleted MatriGel® to the vial and gently pipette the suspension on ice to mix.

Note: It is important to use ice cold media and maintain cells on ice to avoid premature polymerization of the MatriGel®.

Transfer the cell suspension to a 24 well TransWell culture insert, taking care to avoid introduction of air bubbles.

Incubate at 37° C. for 30-45 mins to allow the MatriGel to solidify.

Add 600 μl of pre-warmed growth medium to the Well.

Culture at 37° C. in a 5% CO₂ incubator for 30 days, during which time the media should be changed every 48 hrs.

Note: The culture duration can be altered based on the purpose of the experiment. Longer endpoints are used to study differentiation whereas shorter endpoints of 7 days, 14 days etc., can be used if the purpose of the experiment is not to achieve complete differentiation.

Note: Media was supplemented with Fungizone (0.4%) and Penstrep (1%) for the first 24 hours after seeding and 10 μM Rho kinase inhibitor (Table 4) for the first 72 hrs.

10 μM TGFβ inhibitor (Table 4) was added to the culture media for 15 days to maintain the cells in the proliferative phase and suppress overgrowth of fibroblasts.

Note: Results differ according to culture medium. Results shown herein were generated using PneumaCult™-ALI Medium, which in the Inventors' hands results in generation of large organoids from distal lung, well differentiated and larger organoids from proximal lung.

### Organoid Staining (Table 6):

### Fixing and Embedding of Organoids:

Aspirate the media from the Insert and well of TransWell and rinse once with warm PBS.

Fix the cultures by placing 300 μl PFA (2% w/v) in the Insert and 500 μl in the well for 1 hr at 37° C. Remove fixative and rinse with warm PBS taking care not to dislodge the MatriGel plug.

Note: Fixed organoids can be stored submerged in PBS at 4° C. for one to two weeks before initiating further steps.

Aspirate PBS, invert TransWell and carefully cut TransWell membrane along its periphery. Using forceps, remove transwell membrane, taking care not to disturb the MatriGel plug.

In a petri-dish, tap TransWell to recover the MatriGel plug.

Add a drop of 37° C. HistoGel to the MatriGel plug and maintain at 4° C. until the HistoGel solidifies.

Transfer the HistoGel/MatriGel plug to an embedding cassette, dehydrate through increasing concentrations of ethanol (70, 90 and 100%), clear in xylene and embed in paraffin wax.

Cut 7 μm sections on a microtome and collect on positively charged slides.

### Immunofluorescence Staining of Organoids:

Place slides at 65° C. for 30 minutes to dewax the slides.

Deparaffinize the sections by immersion in xylene and rehydrate through decreasing concentrations of ethanol.

Perform High temperature antigen retrieval in Antigen Unmasking Solution, citric acid base using a Retriever 2100.

Surround the tissue with a hydrophobic barrier using a Pap pen.

Block non-specific staining between the primary antibodies and the tissue, by incubating in Blocking buffer (Table 7).

Incubate sections in the appropriate concentration of primary antibodies (Table 6) diluted in incubation solution (Table 7) overnight at 4° C. in a humidified chamber.

Rinse sections 3 times at room temperature with a washing buffer (Table 7).

Incubate in the appropriate concentration of fluorochrome conjugated secondary antibody for 1 hr at room temperature.

Rinse sections 3 times at room temperature with 0.1% Tween 20-TBS.

Incubate sections for 5 mins in DAPI (1 μg/mL).

Rinse sections once in 0.1% Tween 20-TBS, dry and mount in Fluormount G solution.

Note: Source and optimal working dilution of primary and secondary antibodies used for immunofluorescence staining are included in Table 6.

### Example 2

### Source Lung Tissue

The trachea and extrapulmonary bronchus (FIG. 1A) were used as the source tissue for isolation of proximal airway epithelial cells and subsequent generation of proximal organoids. Distal lung tissue that includes both parenchyma and small airways of less than 2 mm in diameter (FIG. 1A) were used for the isolation of small airway and alveolar epithelial cells (distal lung epithelium) and generation of either small airway or alveolar organoids. Proximal airways lined by a pseudostratified epithelium include abundant basal progenitor cells that are immunoreactive for the membrane protein NGFR (FIGS. 1B and 1C). In contrast, epithelial cells lining alveoli included a subset showing apical membrane immunoreactivity with the HTII-280 monoclonal antibody, suggestive of their alveolar type 2 cell identity (FIGS. 1B and 1D). These surface markers were used to subset single cell suspensions of epithelial cells isolated from either proximal or distal regions.

### Example 3

### Tissue Dissociation and Cell Fractionation

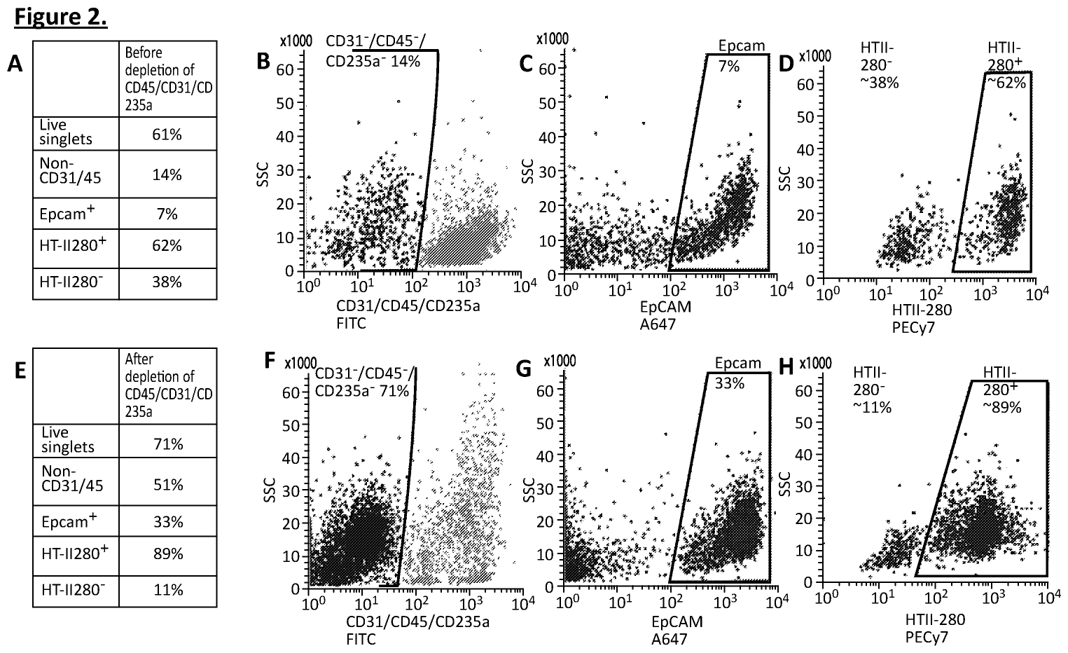

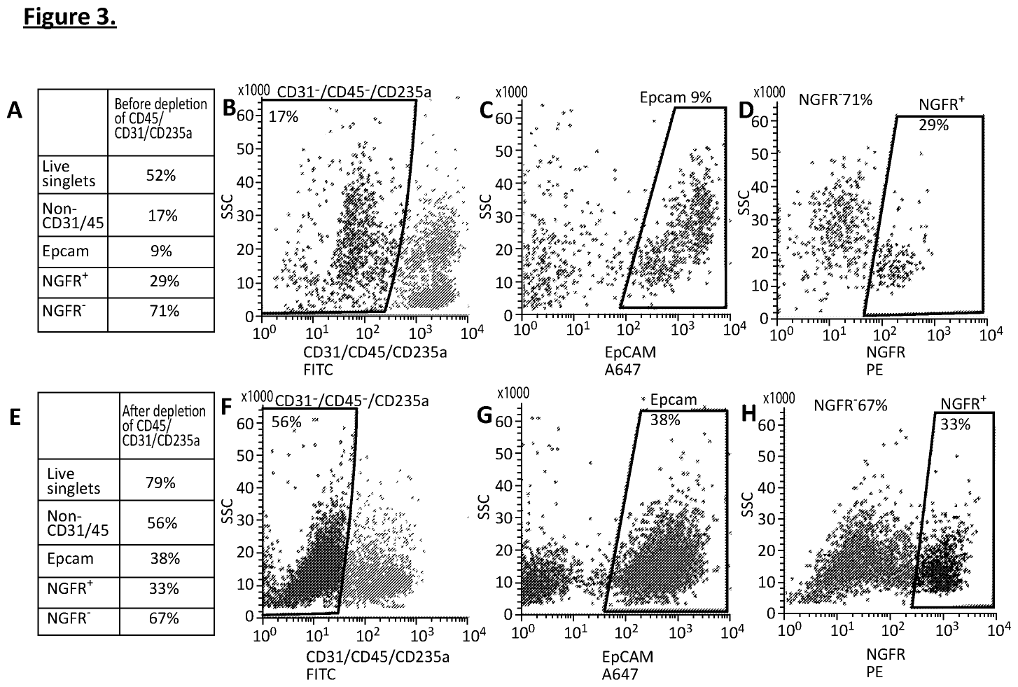

Single cell suspensions of total cells were isolated from either proximal or distal regions of human lung tissue and fractionated using both magnetic bead and FACS to yield enriched epithelial cell populations (FIG. 2 & FIG. 3). Abundant contaminating cell types including red blood cells, immune cells and endothelial cells were stained using antibodies to CD235a, CD45 and CD31, respectively, followed by magnetic-associated cell sorting (MACS) for depletion of these cell types from the total pool of lung cells. The resulting “depleted” cell suspensions were significantly enriched for epithelial cell populations in both distal (FIG. 2E) and proximal (FIG. 3E) tissue samples, with corresponding increase in FACS efficiency. After depletion of CD235a/CD45/CD31 positive cells using MACS the percentage of CD32⁻/CD45⁻/CD235a⁻ increased from 14% (FIG. 2A, 2B) to 51.7% (FIG. 2E, 2F) in distal population. Further FACS depletion of cells staining positively for either CD235a, CD45 or CD31, elimination of cells with positive staining for DAPI and positive selection for the epithelial cell surface marker CD326, led to highly enriched distal cell population that accounted for 33.5% (FIGS. 2E and 2G) compared to 7% (FIG. 2A, 2D) before depletion of negative population. Further subsetting of distal epithelial cell populations was achieved by fractionation based upon surface staining with the HTII-280 monoclonal antibody (FIGS. 2D & 2H), respectively. Accordingly, distal lung epithelial cells included 4.3% HTII-280⁺ and 2.6% HTII-280⁻ subsets (FIG. 2D without depleting of CD31/CD45/CD235a) and 30% HTII-280⁺ and 3.6% HTII-280⁻ subsets (FIG. 2H after depleting of CD31/CD45/CD235a).

Total cells isolated from proximal region were depleted for CD235a/CD45/CD31 positive cells using MACS and the % of CD31⁻/CD45⁻/CD235a⁻ increased from 17% (FIG. 3A, 3B) to 56.6% (FIG. 3E, 3F). Positive selection for the epithelial cell surface marker CD326 in cells isolated from proximal region, led to highly enriched proximal cell population that accounted for 38% (FIGS. 3E and 3G) of total lung cell fractions compared to 9.3% (FIG. 3A,3D) without depletion of negative population respectively. Further subsetting of proximal epithelial cell populations was achieved by fractionation based upon surface staining with antibodies to NGFR (FIG. 3D, 3H), respectively. Accordingly, proximal lung epithelial cells included 2.7% NGFR⁺ and 6.5% NGFR⁻ subsets (FIG. 3D without depleting of CD31/CD45/CD235a) and 13% were NGFR⁺ and 25% NGFR⁻ (FIG. 3H after depleting of CD31/CD45/CD235a).

### Example 4

### Lung Organoid Cultures

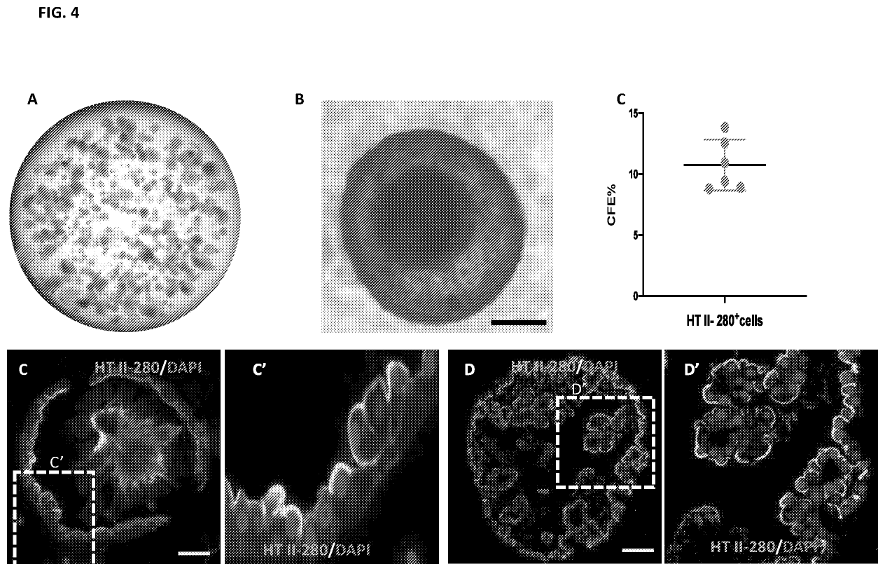

Distal lung epithelial organoids were cultured within growth-factor depleted MatriGel in media that were empirically tested to optimize for organoid growth and differentiation. Three different media were evaluated including PneumaCult™-ALI medium, small airway epithelial cell growth medium (SAECG medium) and mouse Basal medium (media compositions are included in Table 4). Optimal organoid growth was obtained using PneumaCult™-ALI medium, which was selected for further studies. Cultures of HTII-280⁺ distal lung epithelial cells yielded rapidly expanding organoids with an average colony-forming efficiency of 10% (FIG. 4A-4C). Immunofluorescence staining of day 30 cultures using the HTII-280 monoclonal antibody revealed lumen-containing organoids composed predominantly of HTII-280+ distal lung epithelial cells (FIGS. 4D, 4D′ and 4E, 4E′). Cultures of distal lung epithelial HTII-280− cells yielded organoids that were composed of a pseudostratified epithelium resembling that of small airways (not shown).

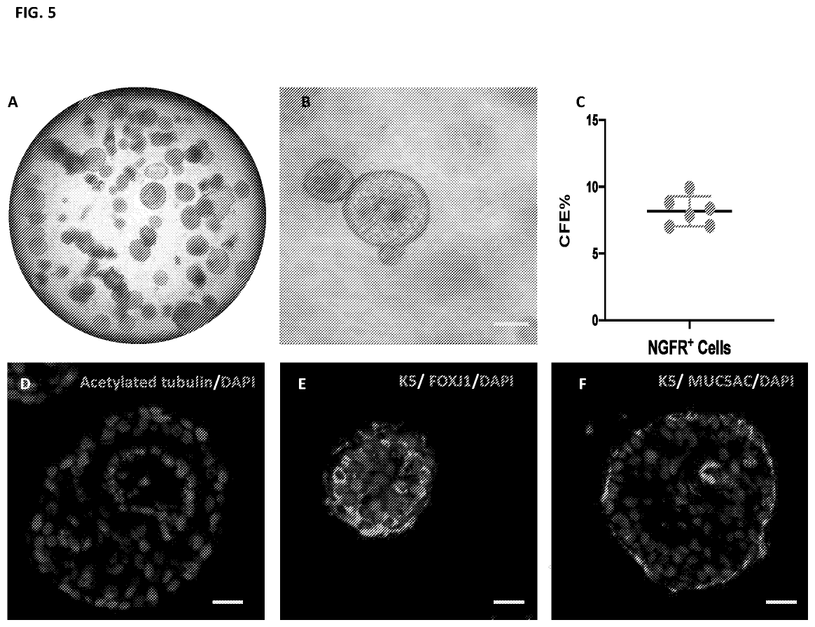

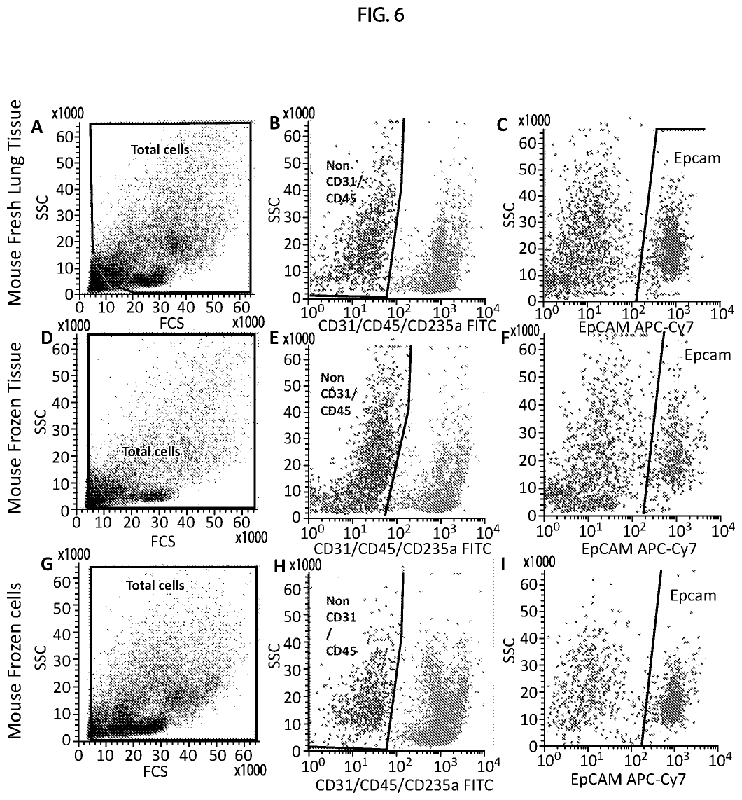

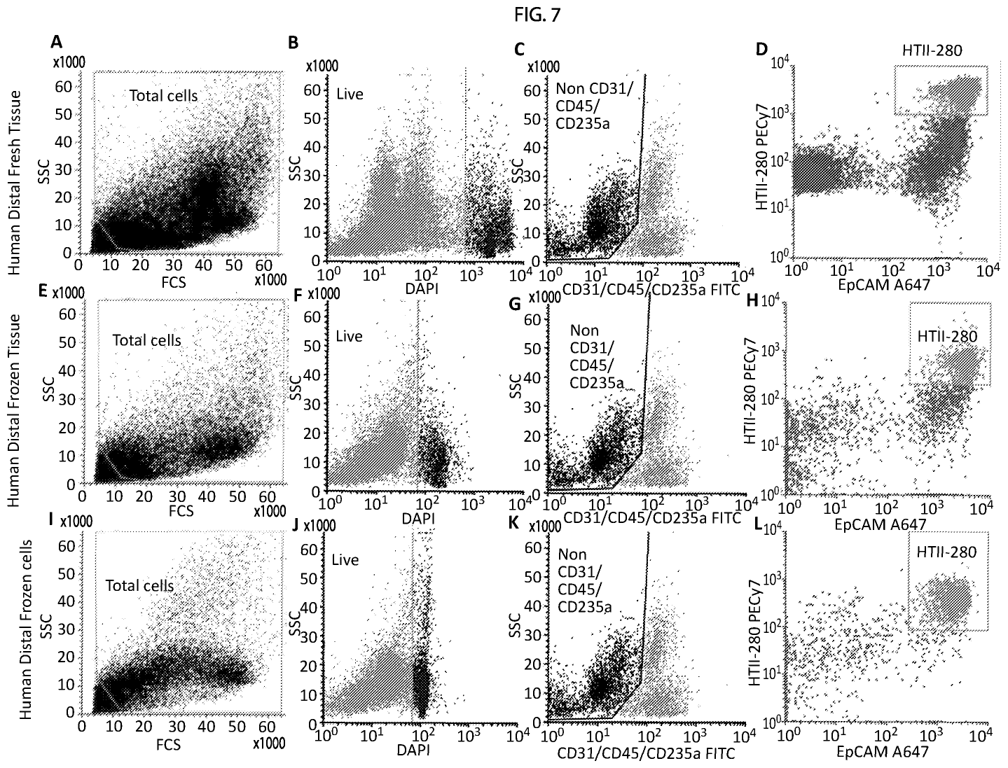

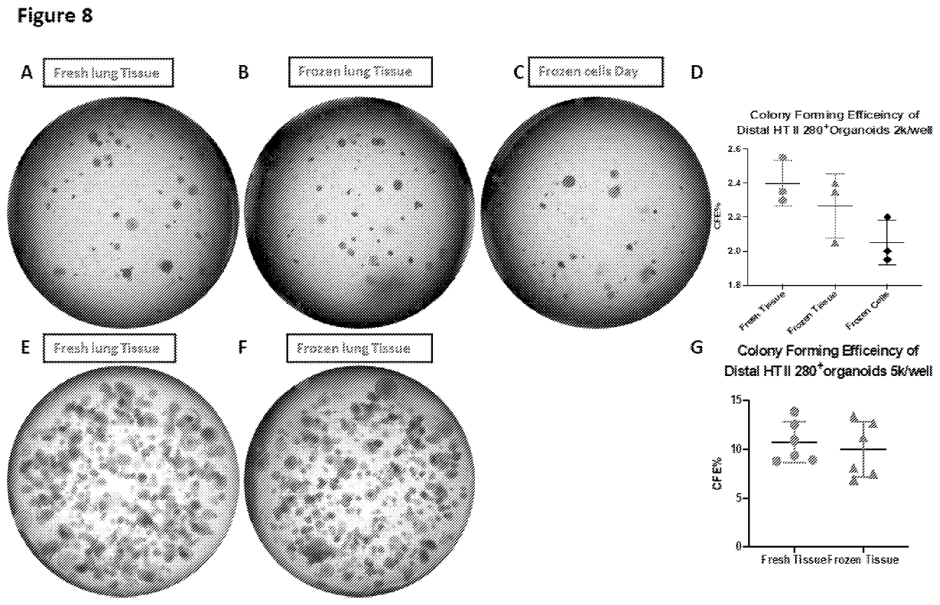

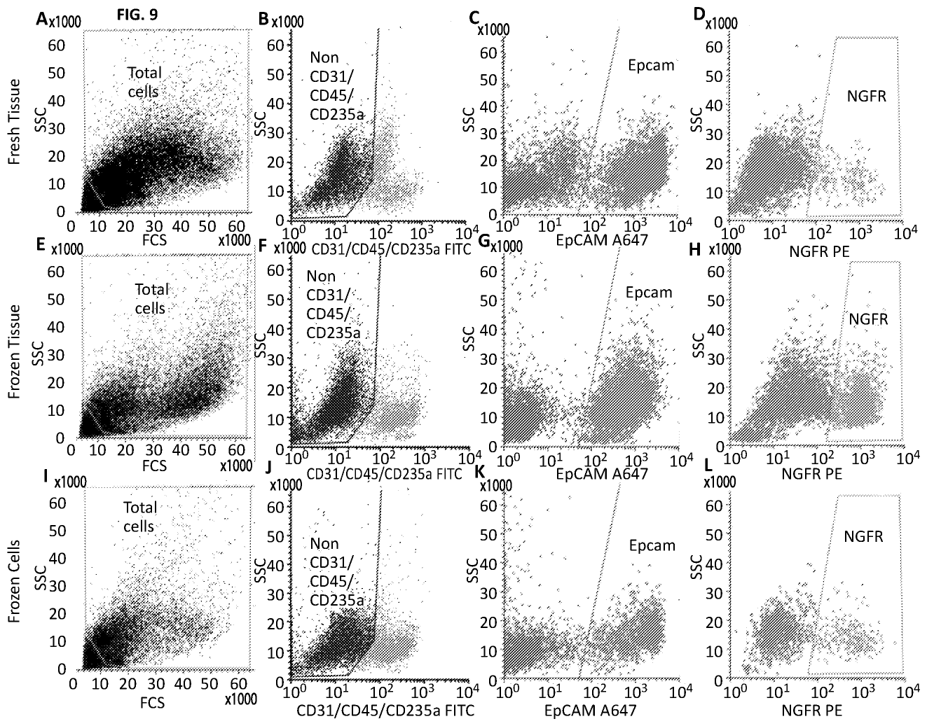

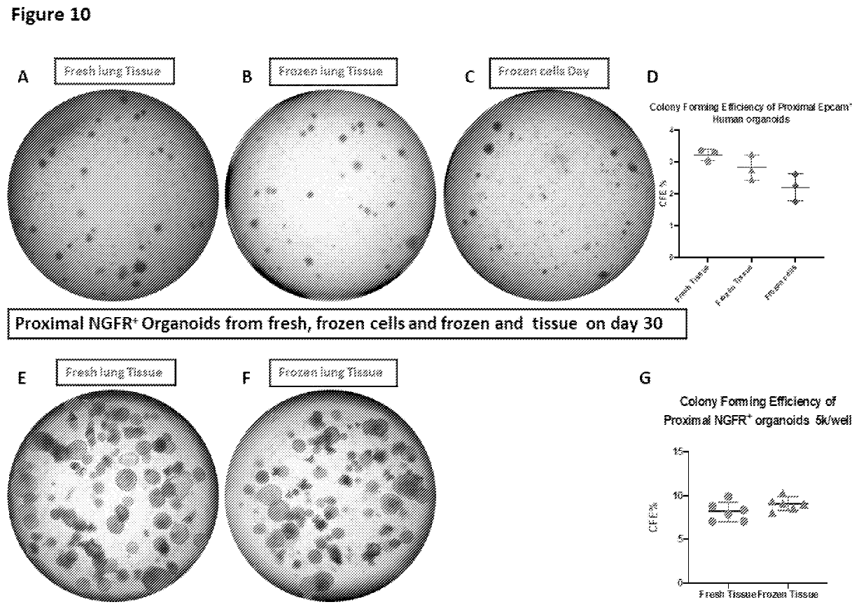

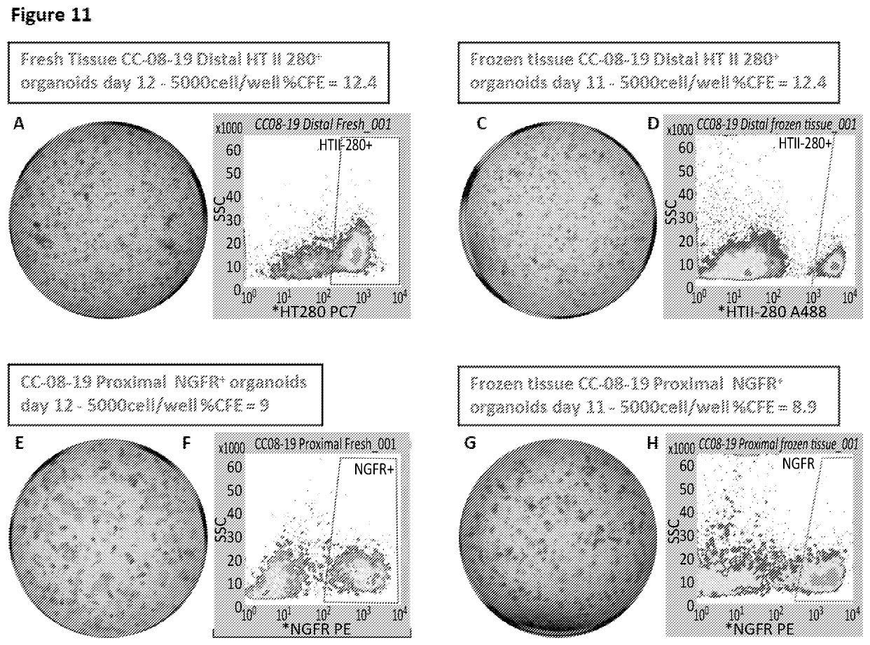

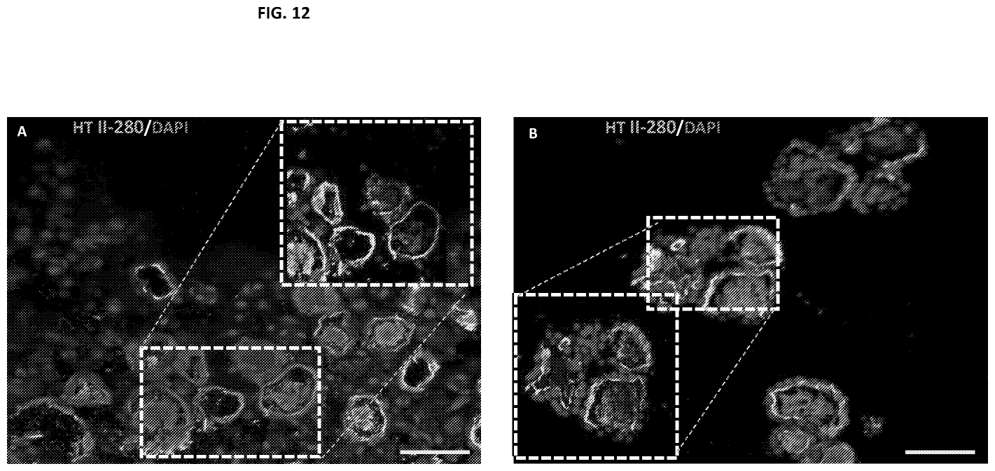

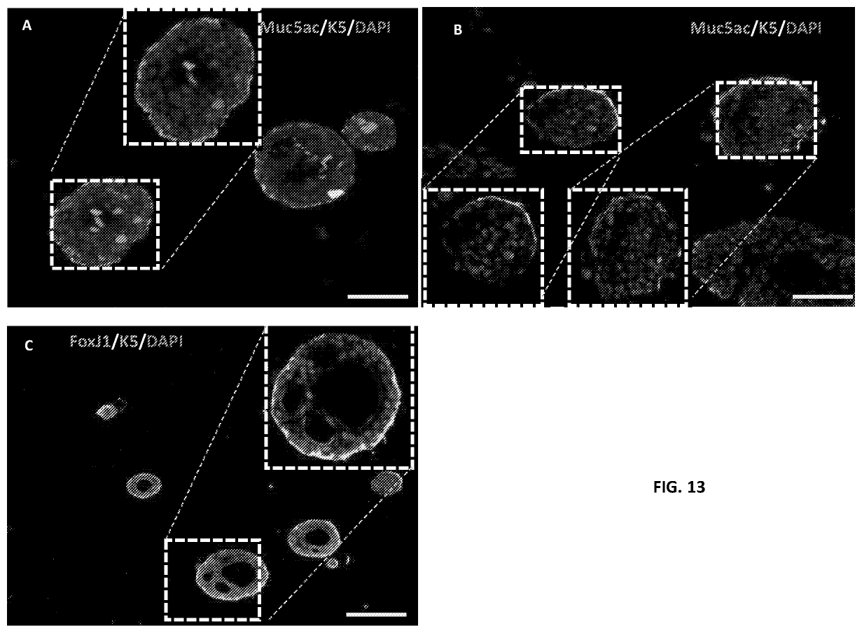

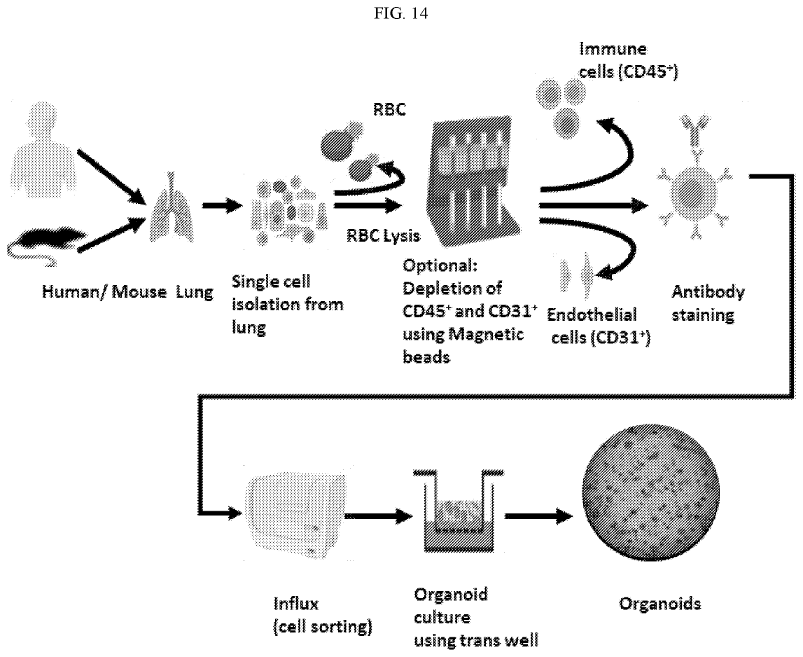

Proximal lung epithelial organoids were cultured from NGFR⁺ cells seeded into MatriGel and cultured for 30 days in PneumaCult™-ALI medium. Large lumen-containing organoids were observed (FIGS. 5A and 5B) with an average colony-forming efficiency of 7.8% (FIG. 5C). Organoids were composed of a pseudostratified epithelium composed of self-renewing Krt5-immunoreactive basal cells and differentiated luminal cell types including AcT+/FOXJ1+ ciliated cells and MUC5AC+ secretory cells (FIG. 5D-F).

### Example 5

### Materials List

TABLE l
Materials for cell isolation

| Cell Isolation | | |
|---|---|---|
| VWR ® Razor Blades | VWR | 55411-050 |
| Falcon® Disposable Petri Dishes, Sterile, Corning ® | VWR | 25373-187 |
| pluriStrainer ® 40 μm (Cell Strainer) | Pluriselect | 43-50040-51 |
| pluriStrainer ® 70 μm (Cell Strainer) | Pluriselect | 43-50070-51 |
| pluriStrainer ® 100 μm (Cell Strainer) | Pluriselect | 43-50100-51 |
| pluriStrainer ® 300 μm (Cell Strainer) | Pluriselect | 43-50300-03 |
| pluriStrainer ® 300 μm (Cell Strainer) | Pluriselect | 43-500500-03 |
| Funnel | Pluriselect | 42-50000-03 |
| connecting ring | Pluriselect | 41-50000-01 |
| Red Blood Cell lysis buffer | eBioscience | 00-4333-57 |
| Liberase ™ TM Research Grade | Sigma Aldrich | 5401127001 |
| HBSS Hank's Balanced Salt Solution 1X 500 ml | VWR | 45000-456 |
| 30 mL Sterile syringes, Luer-Lok Tip | VWR | BD302832 |
| BD Precisionglide needle 16G | VWR | 305198 |
| BD Precisionglide needle 18G | VWR | 305199 |
| Red Biosafety Bags | | |

TABLE 2
Composition of the FACS Buffer

| HBSS Hank's Balanced Salt | | | 500 ml |
|---|---|---|---|
| Solution 1X 500 ml | VWR | 45000-456 | bottle |
| EDTA (0.5M), pH 8.0, | Thermofisher | AM9260G | 500 μl |
| RNase-free | scientific | | |
| HEPES (1M) | Thermofisher | 15630080 | 5 ml |
| | scientific | | |
| Amphotericin B | Thermofisher | 15290018 | 2 ml |
| | scientific | | |
| Penicillin-Streptomycin- | Thermofisher | 15640055 | 5 ml |
| Neomycin (PSN) Antibiotic | scientific | | |
| Mixture | | | |
| Fetal Bovine Serum | Gemini Bio-Products | 100-106 | 10 ml |

TABLE 3
Equipment

| Thermomixer | Eppendorf | 05-412-503 |
|---|---|---|
| GentleMACS Octo Dissociator | MACS Miltenyi | 130-095-937 |
| | Biotec | |
| GentleMACS C Tubes | MACS Miltenyi | 130-096-334 |
| | Biotec | |
| LS Columns | MACS Miltenyi | 130-042-401 |
| | Biotec | |
| Leica ASP 300s Tissue processor | | |

TABLE 4
Composition of Organoid Culture mediums

| | | | |
|---|---|---|---|
| ThinCert ™ Tissue Culture Inserts, Sterile | Greiner Bio-One | 662641 | |
| PneumaCult ™- ALI Medium | StemcellTechnologies | 5001 | |
| Small Airway Epithelial cell Growth Medium | PromoCell | C-21170 | |
| Y-27632 (ROCK inhibitor) | Stemcell Technologies | 72302 | |
| 100 mM stock (1000x) | | | |
| Mouse Basal medium: | | | |
| DMEM/F-12, HEPES | ThermoFisher scientific | 11330032 | 50 ml |
| Insulin-Transferrin-Selenium (ITS -G) (100X) | ThermoFisher scientific | 41400045 | 500 μl |
| Amphotericin B | Thermofisher scientific | 15290018 | 50 μl |
| Penicillin-Streptomycin- Neomycin(PSN) | Thermofisher scientific | 15640055 | 500 μl |
| Antibiotic Mixture | | | |
| Fetal Bovine Serum | Gemini Bio-Products | 100-106 | 5 ml |
| SB431542 TGF-β pathway inhibitor (stock 10 | Stem cell | 72234 | 5 μl |
| mM) | | | |

TABLE 5
List of antibodies for FACS

| | | | |
|---|---|---|---|
| Mouse IgM anti human HT2-280 | Terrace Biotech | TB-27AHT2-280 | 1:300 |
| FITC anti-human CD31 | BioLegend | 303104 | 1:100 |
| FITC anti-human CD45 | BioLegend | 304054 | 1:100 |
| FITC anti-human CD235a | BioLegend | 349104 | 1:100 |
| Alexa Fluor ® 647 anti-human CD326 | BioLegend | 369820 | 1:50 |
| (EpCAM) Antibody | | | |
| PE anti-human CD271(NGFR) | BioLegend | 345106 | 1:50 |
| CD31 MicroBead | Miltenyi Biotec | 130-091-935 | 20 μl/10⁷ |
| Kit, human | | | total cells |
| CD45 MicroBeads, | Miltenyi Biotec | 130-045-801 | 20 μl/10⁷ |
| human | | | total cells |
| DAPI | Sigma Aldrich | D9542-10MG | 1:10000 |

TABLE 6
List of antibodies for immunofluorescence

| | | | |
|---|---|---|---|
| Histogel | Thermo Scientific | HG-4000-012 | |
| Primary Antibodies | | | |
| Anti HT2-280 | Terracebiotech | TB-27AHT2-280 | 1:500 |
| Keratin 5 Polyclonal Chicken Antibody, | Biolegend | 905901 | 1:500 |
| Purified [Poly9059] 100 μl | | | |
| PDPN/Podoplanin Antibody (clone 8.1.1) | LifeSpan Biosciences | LS-C143022-100 | 1:300 |
| MUC5AC Monoclonal Antibody (45M1) | ThermoFisher Scientific | MA5-12178 | 1:300 |
| Sox-2 Antibody | Santa Cruz | sc-365964 | 1:300 |
| | biotechnologies | | |
| FOXJ1 Monoclonal Antibody (2A5) | Thermo Fisher Scientific | 14-9965-82 | 1:300 |
| Purified Mouse Anti-E-Cadherin | BD biosciences | 610182 | 1:1000 |
| Human Uteroglobin/ SCGB1A1 Antibody | R and D systems | MAB4218 | 1:300 |
| Secondary Antibodies | | | |
| FITC anti-mouse IgM Antibody | BioLegend | 406506 | 1:500 |
| Goat anti-Hamster IgG (H + L), | Thermo Fisher Scientific | A-21113 | 1:500 |
| Alexa Fluor 594 | | | |
| Goat anti-Mouse IgG2a Cross-Adsorbed | Thermo Fisher Scientific | A-21131 | 1:500 |
| Secondary Antibody, Alexa Fluor 488 | | | |
| Goat anti-Mouse IgG2a Cross-Adsorbed | Thermo Fisher Scientific | A-21134 | 1:500 |
| Secondary Antibody, Alexa Fluor 568 | | | |
| Goat anti-Mouse IgG2b Cross-Adsorbed | Thermo Fisher Scientific | A-21144 | 1:500 |
| Secondary Antibody, Alexa Fluor 568 | | | |
| Donkey anti-rabbit IgG, 488 | Thermo Fisher Scientific | A-21206 | 1:500 |
| Goat anti-Mouse IgG1 Cross-Adsorbed | Thermo Fisher Scientific | A-21121 | 1:500 |
| Secondary Antibody, Alexa Fluor 488 | | | |

### Example 6

### Advantages of Cryobanked Human Lung Tissue

Frozen lung tissue provides better logistical flexibility and can be shipped like frozen cells for analysis at different sites.

Lung samples collected from different patients at different time points can be cryobanked for simultaneous processing for either genomic or functional analysis.

Viability of cells isolated following application of this cryobanking protocol is comparable that seen with fresh tissue.

### Example 7

### Protocols

Human lung tissue was obtained from deceased tissue donors in compliance with consent procedures developed by International Institute for the Advancement of Medicine (IIAM) and approved by the Cedars-Sinai Medical Center Internal Review Board.

### Freezing Distal and Proximal Regions of the Lung:

Prepare and autoclave all dissection instruments, glassware and the appropriate solutions one day prior to cell isolation.

Upon receiving lung tissue identify and separate the proximal and distal regions and mince the tissue as described in Konda et al 2020 (refer the first article here). Briefly, dice tissue into approximately 1 cm3 pieces and place in a clean 50 mL tube. Wash tissue three times with chilled HBSS, discarding the HBSS wash each time to remove blood and epithelial lining fluid.

Place tissue in a new petri dish (150×15 mm) and blot dry with sterile Kim wipes. Using forceps and scissors, remove as much visceral pleura as possible. Use scissors to mince tissue into pieces of approximately 3-4 mm diameter.

Add approximately 1 to 1.5 grams of tissue to a 2 mL cryovial and add 1 mL of the cryoprotective media, CryoStor (Table 1).

Label tubes and move the vials to a cell freezing container. Fill the freezing container with isopropyl alcohol place them in −80 degrees overnight.

Transfer the frozen vials to the vapor phase of a liquid nitrogen vessel and record the locations.

Note: Ensure that the tissue is completely submerged in freezing medium in order to prevent formation of ice crystals during the freezing process and prevent tissue damage.

Enrichment and Sub Setting of Epithelial Progenitor Cells from Fresh Lung Tissue

Enrichment and fractionation of the epithelial, small airway and alveolar epithelial progenitor cells from distal lung tissue or tracheobronchial basal progenitor cells from proximal lung tissue using appropriate cell surface markers as described in Konda et al 2020

Enrichment and Sub Setting of Epithelial Progenitor Cells from Frozen Lung Tissue

Thaw frozen proximal or distal tissue by placing the vial at 37 degrees for 1-2 mins.

Transfer the tissue to a sterile Petri dish, add 5 ml of FACS buffer (Table 1) to the tissue and allow the tissue to equilibrate in an incubator at 37° C. for 10 mins.

Transfer the contents into a sterile 50 ml tube and centrifuge at 500×g for 5 mins.

Aspirate the supernatant and move the tissue to a new sterile Petri dish. Finely mince the tissue using single sided razor blade and perform enzymatic digestion to obtain a single cell suspension of total cell as mentioned in Konda et al 2020.

Deplete CD31+ endothelial cells and CD45+ immune cells from the pool of total cells using the Milteny MACS CD31 & CD45 microbeads conjugated to monoclonal anti-human CD31 and CD45 antibody (isotype mouse IgG1) and LS columns in accordance to the manufacturer's protocol.

Collect flow through consisting primarily of epithelial and stromal cells in a fresh sterile tube and centrifuge it at 600×g for 5 mins at 4° C. Perform a cell count to ascertain the total number of cells in the flow through

### Cell Surface Staining for Fluorescence Associated Cell Sorting (FACS) in Both Proximal and Distal Lung

Resuspend 1×10⁷ cells per 1 mL of HBSS+ buffer. Add primary antibodies at the required concentration and incubate the cells for 30 mins at 4° C. in dark. Details of antibody sources and titers are described in Konda et al 2020.

Wash off excess antibody by adding 3 mL of HBSS+ buffer and centrifuge at 600×g for 5 mins at 4° C.

Discard supernatant and resuspend cells in HBSS+ buffer per 1×10⁷ cells/mL.

Filter cells into 5 mL polystyrene tubes through a strainer cap to ensure formation of a single cell suspension.

Add DAPI (1 μg/mL) to stain permeable (dead) cells.

### Organoid Culture and Staining.

The protocol for organoid culture and staining is explained in detail in Konda et al 2020. Mention how many cells you seed per transwell.

### 10×RNA Sequencing Experiment.

Capture the CD45-CD31-CD326+ lung epithelial cells from fresh and frozen tissue using a 10× Chromium device (10× Genomics) and prepare libraries according to the Single Cell 3′ v2 Reagent Kits User Guide (10× Genomics).

Quantify the barcoded sequencing libraries by quantitative PCR using the KAPA Library Quantification Kit (KAPA Biosystems, Wilmington, Mass.).

Sequenced the libraries using Novaseq 6000 (Illumina)

With the above approaches, the Inventors clearly defined proximal and distal regions of the lung with the goal of isolating region-specific progenitor cells. The Inventors utilized a combination of enzymatic and mechanical dissociation to isolate total cells from the lung and trachea. The Inventors then fractionated specific progenitor cell from the proximal or distal origin cells using Fluorescence associated cell sorting (FACS) based on cell type specific surface markers, such as NGFR for sorting basal cells and HTII280 for sorting alveolar type II cells. Isolated basal or alveolar type II progenitors were used to generate 3D organoid cultures. Both proximal and distal progenitors formed organoids with a colony forming efficiency of 9-13% in distal region and 7-10% in proximal region when plated 5000 cell/well on day 30. Distal organoids maintained HTII-280⁺ alveolar type II cells in culture whereas proximal organoids differentiated into ciliated and secretory cells by day 30. These 3D organoid cultures can be used as a robust experimental tool for studying the cell biology of lung epithelium and epithelial mesenchymal interactions, as well as for and validating therapeutic strategies.

### Example 8

### Animals

Experiments were performed with specific pathogen free C57Bl/6 mice in accordance with institutional IACUC protocol.

### Human Lung Samples

Human lung tissue was obtained from deceased tissue donors in compliance with consent procedures developed by the International Institute for the Advancement of Medicine (IIAM) and approved by the Cedars-Sinai Medical Center Internal Review Board.

### Freezing Distal Regions of the Lung:

Upon receiving lung tissue, identified and separated distal regions and briefly, diced tissue into approximately 1 cm3 pieces and placed in a clean 50 mL conical tube. Washed tissue to remove blood and epithelial lining fluid and minced the tissue into pieces of approximately 3-4 mm diameter. Added approximately 1 to 1.5 grams of tissue to a 2 mL cryovial and added 1 mL of the cryopreservative media, CryoStor. Moved the vials to a cell freezing container. Filled the freezing container with isopropyl alcohol and placed it in a −80-degree freezer overnight. Transferred the frozen vials to the vapor phase of a liquid nitrogen vessel.

Note: Ensure that the tissue is completely submerged in freezing medium in order to prevent the formation of ice crystals and tissue damage during the freezing process.

Enrichment and Sub Setting of Epithelial Progenitor Cells from Fresh or Frozen Lung Tissue.

Enriched and fractionated the epithelial, small airway and alveolar epithelial progenitor cells from distal lung tissue using appropriate cell surface markers, as previously described (JoVe-REF).

Thawed previously frozen distal tissue by placing the vial at 37 degrees for 1-2 mins. Transferred the contents into a sterile 50 ml tube and centrifuged by adding 10 ml of HBSS buffer at 500×g for 5 mins.

Aspirated the supernatant and moved the tissue into a new sterile Petri dish. Finely minced the tissue using single sided razor blade and performed enzymatic digestion using Liberace and Dispase to obtain a single cell suspension of total cells, depleted CD31+ and CD45+ cells and performed surface staining for fluoresce associated cell sorting (FACS) as mentioned in Konda et al 2020. Enrichment of epithelial progenitor cells from frozen cells also performed in a similar way. Collected the HT 11-280+ cells and proceed with culturing organoids.

### Proliferation of Organoid on Large Scale for Drug Screening Studies.

Removed the media from bottom well of the trans well and washed once with PBS. Dissolve the Matrigel® by using Corning cell recovery solution and followed the instruction form Corning. Collected the cells in FACS buffer (supplemental data) and centrifuged it at 600×g for 5 mins at 4° C. Aspirated the buffer and proceeded for FACS staining. Collected HT II 280⁺ cells from the sorter and recounted them to make sure the accurate cell count. Mixed 5000 HT II 280⁺ cells 7.5×10⁴ MRC-5 cells (human lung fibroblast cell line) and proceeded with organoid culture. Each confluent well can be passaged to 4-6 wells and we can repeat passage couple of times until we get desired yield for the experiments. We passaged organoids 3-4 times and used for different experiments such as studying infection of primary human lung epithelium for disease modeling, and drug discovery.

Isolation of Cells from Mouse Lung.

Detailed description of the extracting the lungs from mouse, dissociation of mouse lung tissue for single cell suspension, depletion of negative population and cell sorting are available in the online supplement.

10×RNA Sequencing Experiment

Captured the CD45⁻, CD31⁻, CD326⁺ lung epithelial cells from fresh and frozen tissue. Libraries were prepared according to the Single Cell 3′ v2 or v3 reagent kits user guide (10× Genomics). Cellular suspensions were loaded on a Chromium Controller instrument (10× Genomics) to generate single-cell Gel Bead-In-Emulsions (GEMs). Reverse transcription (RT) was performed in a Veriti 96-well thermal cycler. The barcoded sequencing libraries were quantified by quantitative PCR using the KAPA Library Quantification Kit (KAPA Biosystems, Wilmington, Mass.). Sequencing libraries were loaded on a NovaSeq 6000 (Illumina). Cell Ranger software (10× Genomics) was used for mapping and barcode filtering. Briefly, the raw reads were aligned to the transcriptome using STAR(5), using a hg38 transcriptome reference from Ensemble 93 annotation. Expression counts for each gene in all samples were collapsed and normalized to unique molecular identifier (UMI) counts. Data analysis was performed with Conos, a recently established tool that was developed for joint analysis of heterogeneous datasets. Data have been deposited under GEO.

### Mouse Lung Epcam⁺ Cell Fractionation Comparison Fresh Tissue Vs Frozen Tissue Vs Frozen Cells:

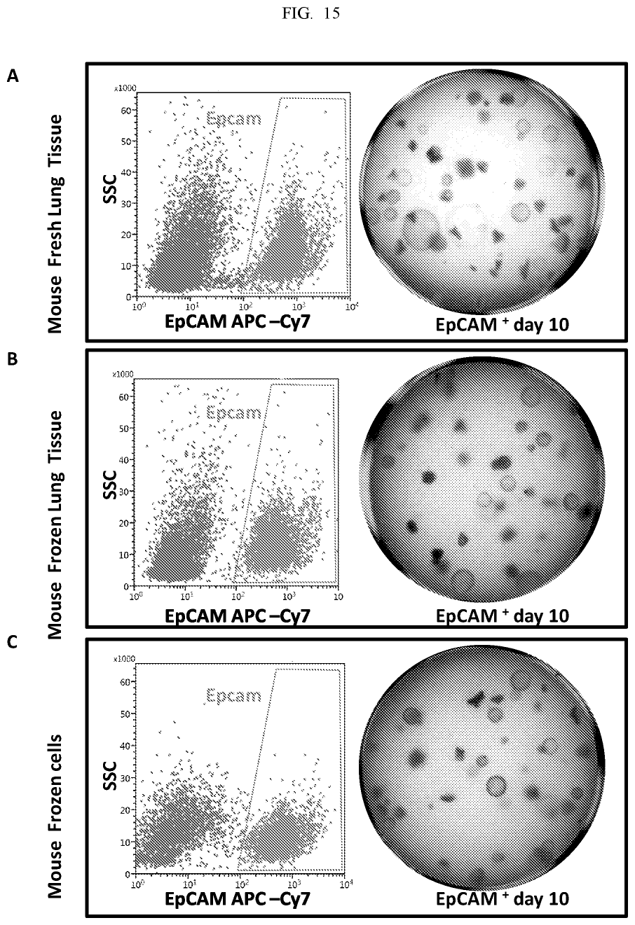

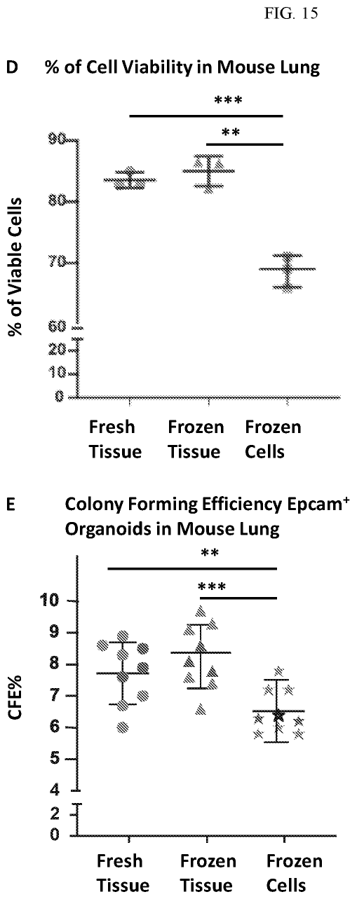

FACS analysis was performed on the following samples—cells freshly isolated from the mouse lungs (FIG. 15A), cells isolated from cryobanked pieces of mouse lung tissue (FIG. 15B), and cryobanked single cell suspensions of mouse lung cells recovered after freezing (FIG. 15C). For all conditions, epithelial cells were identified based on exclusion of CD31⁺ endothelial cells and CD45⁺ immune/hematopoietic cells, followed by positive selection for EpCAM (CD326). We observed comparable distributions of Epcam+ populations in all samples. Colony forming efficiency was similar in both fresh and frozen tissue and P values is non-significant between these two groups (FIG. 15E). P value is 0.0007 (***) between Frozen mouse lung cells and Frozen mouse lung tissue and P value is 0.0089 (**) between Frozen mouse lung cells and Fresh mouse lung tissue. Similar % of cell viability is similar between fresh mouse lung and frozen mouse lung tissue and P value was non-significant between these two groups (FIG. 15D). P value is 0.0008 (***) between Frozen mouse lung cells and fresh mouse tissue and P value is 0.0013 (**) between Frozen mouse lung cells and frozen mouse lung tissue.

### Human Distal Lung Cell Fractionation Comparison Fresh Tissue Vs Frozen Tissue Vs Frozen Cells:

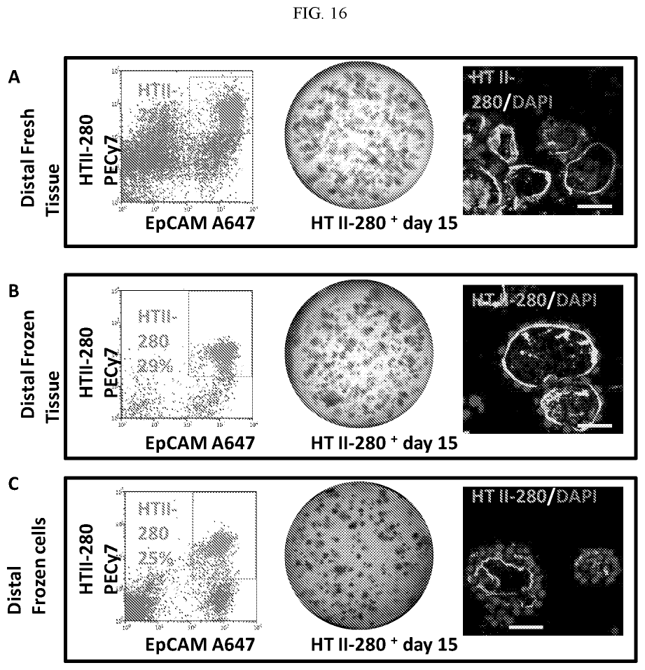

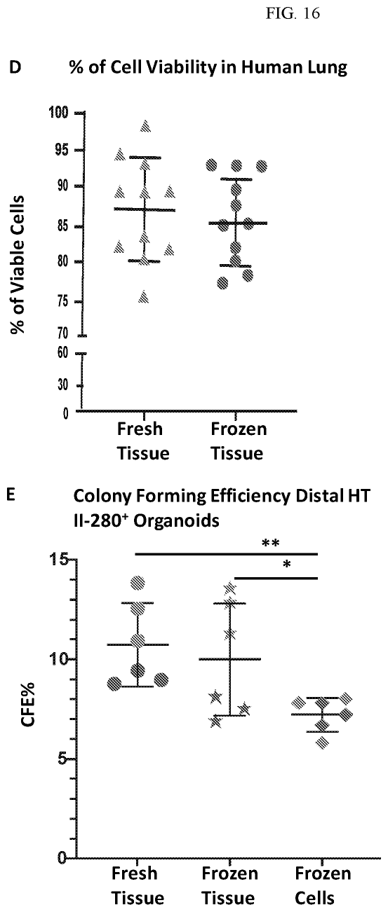

CD326+Epithelial cells harvested directly from human lung explants (“fresh”, FIG. 16A), from frozen human lung tissue pieces (FIG. 16B), and frozen dissociated human lung cells (FIG. 16C) were isolated by FACS as mentioned above. Abundant contaminating cell types including immune cells, red blood cells and endothelial cells were stained using antibodies to CD45, CD235a and CD31, respectively, followed by magnetic-associated cell sorting for depletion of these cell types from the total pool of cells. The resulting “depleted” cell suspensions were significantly enriched for epithelial cell populations. Further FACS depletion of cells staining positively for either CD45, CD235a and CD31, elimination of cells with positive staining for DAPI and positive selection for the epithelial cell surface marker CD326, led to highly enriched distal cell population. Additionally, lung epithelial cells were further sub fractionated by their expression of a Type II-specific antigen, HTII-280. The percentage of HT II 280+positive calculation was performed on an average of 4 different biological samples in all the three groups (A, B, C). We compared viability of cells isolated from fresh tissue and frozen tissue (FIG. 16D) and there is no significant P value between these two groups. HT II 280+ cultures formed rapidly expanding organoids, with an average CFE of 10% in both fresh and frozen distal tissue. The % CFE with no significant P value between fresh and frozen human lung tissue (FIG. 16E). In comparison the % CFE of organoids generated from frozen single cells was significantly lower when compared to both fresh and frozen tissue (FIG. 16E) with a slightly lower amount. Distal lung epithelial organoids from fresh tissue (FIG. 16A), frozen tissue (FIG. 16B) and frozen cells (FIG. 16C) were cultured with growth-factor depleted Matrigel® in Pneumacult ALI medium. In all three conditions, the organoid sizes were consistent, while immunofluorescence staining of organoids revealed organoids with lumen and positive staining for HT II 280 antibody. Our novel cryopreservation technique helps in preserving % CFE indicative of the survival and self-renewal capacity of HT II 280+ progenitors at the same level as freshly dissociated cells. Whereas traditionally used method (freezing cell suspension) has significantly lower.

Transcriptomic Comparison of Datasets Derived from Freshly Isolated and Frozen Tissue:

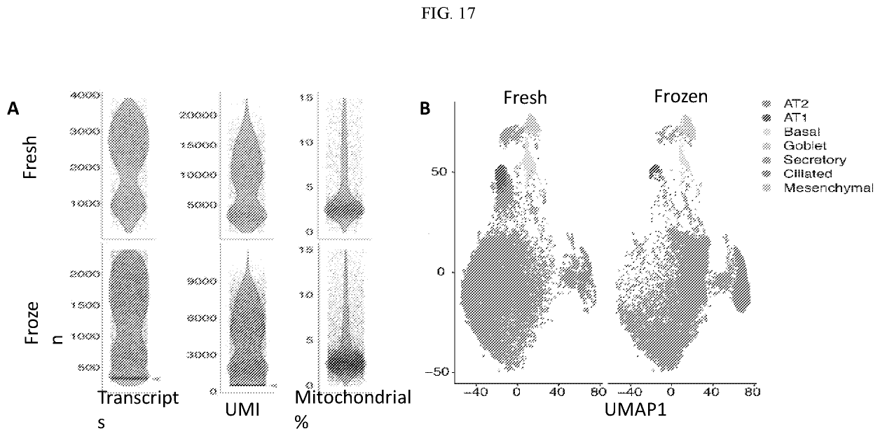

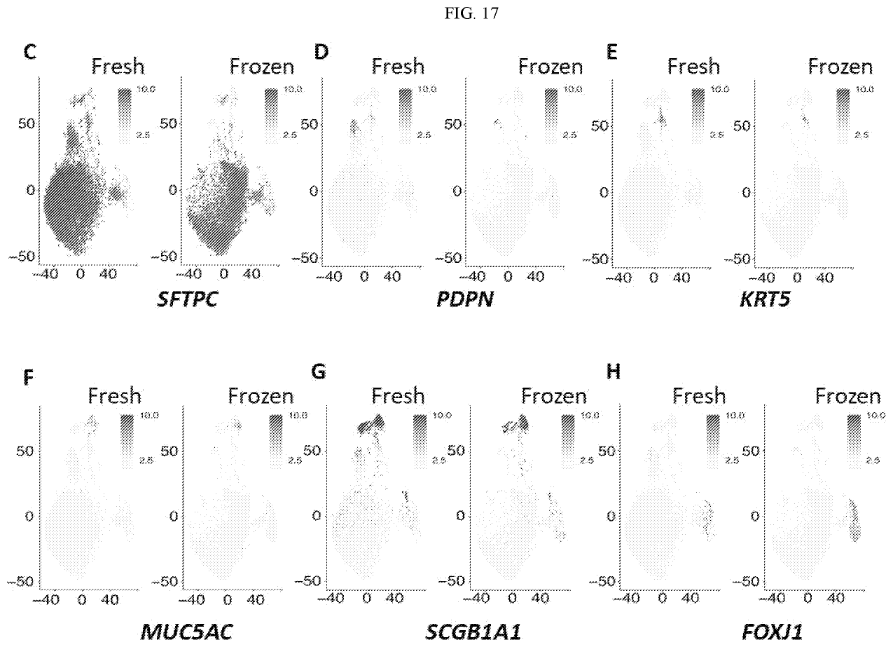

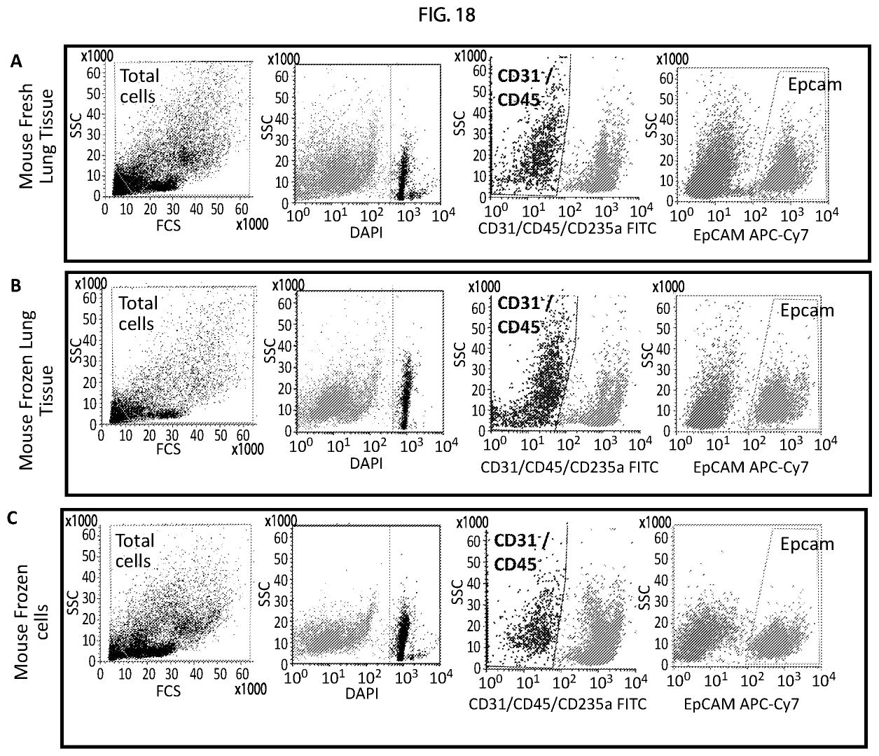

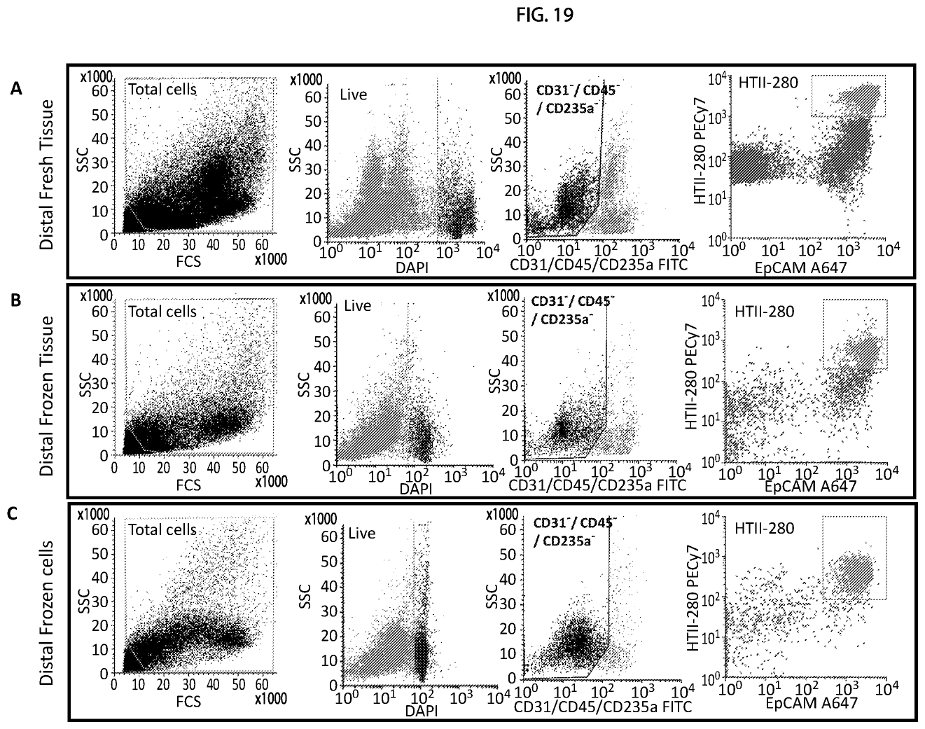

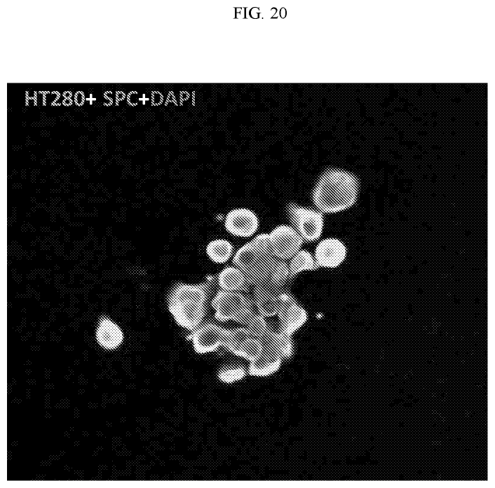

To assess the quality of transcriptomic data derived from processing of frozen tissue, we performed scRNAseq analysis, side by side on cells derived from frozen and freshly isolated tissue. Analysis of sequencing quality showed comparable metrics, including sequencing saturation, number of reads mapped to exons, and number of genes per cell (Table 1). Following quality control and filtering, ‘fresh’ and ‘frozen’ datasets showed similar distribution for number of transcripts, UMIs, and percentage of mitochondrial genes. Of note, in the dataset derived from frozen tissue an accumulation of cells with low number of transcripts and UMI was observed, suggesting a specific effect derived from tissue cryopreservation (FIG. 17A). Major cells types, detected in the dataset derived from freshly isolated tissue, were also observed in the cryopreserved counterpart (FIG. 17B-H). This result suggests that cryopreservation of tissue with the protocol here presented is a valuable method to store rare and precious specimens and that this material can be subsequently successfully used for single cell transcriptomic studies.

### Example 9

Extracting Lung from Mouse:

After anesthetizing the mouse, using i.p. Ketamine and Xylazine, it was placed in surgical plane in biosafety cabinet. It was pinned using 5-point method. The mouse was then dampened with 70% ETOH. Its abdominal cavity was opened, and it was exsanguinated via a transection of inferior vena cava. Deflated the lungs by nicking the diaphragm and cut diaphragm along the bottom of the rib cage. Opened thoracic cavity on left side and cut under left clavicle to reveal trachea. Blood around trachea was cleaned up with gauze and the lung was perfused with 10 ml of 1×PBS. Small incision was made just below the larynx between cartilage rings using fine scissors, and then cannulated trachea by inserting a BD Insyte Autoguard. Tied the catheter and trachea together using black surgical string. Cannula cannot be allowed to pass bifurcation. Performed bronchoalveolar lung lavage by attaching 1 ml syringe to Autoguard and washed the lung with saline three time by injecting and aspirating PBS 3-4 times. Extracted the lung along with heart and cannula from the mouse. Immersed the tissue in ice cold sterile 1×PBS in 50 ml conical test tube. Stored conical tube on ice until lungs are ready for elastase digestion.

### Mouse Lung Tissue Dissociation (Preparing Single Cell Suspension)

Prepared working solution of Liberase™ (Roche) at 0.25 Wünsch U/ml in HBSS (4 ml per 1 mouse whole lung tissue) and warm to 37° C. Note: Liberase stock solution is a 100× (i.e., 25 Wünsch U/ml). Calculated stock concentration of Elastase (U/ml). Concentration of stock enzyme will vary lot to lot. Added the necessary volume of the enzyme to get to 4 U/ml (for example, if we have 3 mice, we need 9 ml of elastase solution. 10 ml of the solutions need to be made. If stock elastase concentration is 120 U/ml, 40 units, or 334 μl, need to be added to 9666 μl Ham's F12). This suspension will be cloudy. Dissolved the enzyme into the Hams F12. Adjusted the pH of the solution up using 2N NaoH to 8.5 or 9 or until the solution starts clearing up. Make sure pH don't go over 10, as this might denature the enzyme. Once the solution was clear, added the necessary amount of 2N HCl to bring the pH back down to 7.4. Note: keeping the medium with enzyme in 37° C. water bath may not require the base/acid pH adjustment. Make sure the solution is clear and pH is at 7.0 before using it. Filled a 3 ml syringe with the 4 U/ml elastase solution. Instilled 1 ml elastase into each lung, keeping syringe attached to cannula, incubated for 10 minutes. Repeated 4 times with 0.5 ml elastase and an incubation period of 5 minutes. Removed the digested tissue and placed it on the Petri dish. Diced lung sample into smaller pieces on a Petri dish and disaggregate the tissue by mechanical mincing with a single sided razor blade or scissors. Moved the tissue into new clean 50 ml tube. Note: Add warmed (37° C.) Liberase™ solution to the tube (4 ml of HBSS+40 ul 100× Liberase™ per 1 mouse). Placed the tube in a Thermomixer (Eppendorf) and agitated at 1000 rpm at 37° C. for 60 min. (Set Thermomixer time=45 minutes). Triturated the tissue up/down using 10 ml syringe. Strained the lung digest through 70 μm strainer into new 50 ml conical to remove the extracellular matrix. After straining, washed the filters with 10 ml of HBSS-FACS buffer. Resuspend the lung digests in HBSS-FACS buffer and centrifuged at 400 g for 5 mins at 4° C. Decanted the supernatant and resuspend the cell pellet in 1 ml of Red Blood Cell (RBC) lysis buffer and incubate for 1.5 mins with gentle agitation at RT. (rock back/forth in hands). Immediately added 20 ml HBSS-FACS buffer to dilute the lysis buffer. Strained the solution through 70 μm strainer into new 50 ml conical to remove RBC ghost cells. Centrifuged the cells at 400 g for 5 mins at 4° C.; aspirated the supernatant and resuspend the cells in an appropriate volume of HBSS-FACS buffer for cell counting. Counted the cells (used a hemocytometer).

### Depletion of Negative Population:

Resuspended the cells in 90 μl of HBSS-FACS buffer per 10⁷ total cells. Added 10 μl of CD45 Microbeads and 10 μl of CD31 Microbeads. Mixed the cells and incubate for 15-20 minutes in the refrigerator (2-8° C.). Washed the cells by adding 1-2 mL of HBSS-FACS buffer per 10⁷ cells and centrifuged at 300×g for 5 minutes. Aspirated supernatant completely. Placed LS Column in the magnetic field of a suitable MACS Separator. Prepared the column by rinsing with 3 mL of HBSS-FACS buffer. Added cell suspension onto the column. Collected the unlabeled cells that pass through. Performed three washing steps with 3 mL of HBSS-FACS buffer each. Collected total effluent; this is the CD45− and CD31− fraction. Proceed with cell sorting.

### Cell Surface Staining for Fluorescence Associated Cell Sorting (FACS) in Mouse Lung

Resuspend 1×10⁷ cells per 1 mL of HBSS+ buffer. Added primary antibodies at the required concentration and incubated the cells for 30 mins at 4° C. in dark. In this study, fluorophore conjugated primary antibodies were used unless otherwise stated. Details of antibody sources and titers are described in table. Washed the excess antibody by adding 3 mL of HBSS+ buffer and centrifuged at 600×g for 5 mins at 4° C.

Note: If using unconjugated primary antibodies, add required concentration of an appropriate fluorophore conjugated secondary antibody and incubate for 30 mins on ice.

Washed off excess secondary antibody by adding 3 mL of HBSS+ buffer and centrifuged at 600×g for 5 mins at 4° C. Discarded the supernatant and resuspend cells in HBSS+buffer per 1×10⁷ cells/ml. Filtered the cells into 5 mL polystyrene tubes through a strainer cap to ensure formation of a single cell suspension. Added DAPI (1 μg/mL) to stain permeable (dead) cells. Cells are sorted on Influx.

### Example 10

### Platforms to Test Viral Infection Using Organoids

3D organoid culture: organoids may be directly tested with viral infection or drug screening

Regional lung organoid models—distal human lung organoids: unfractionated distal lung epithelial cells organoids. Distal organoids are infected either in situ or after Dispase (protease which cleaves fibronectin, collagen IV, and to a lesser extent collagen I) treatment to remove the Matrigel®.

Regional lung organoid models—proximal human lung organoids: using epithelial cells isolated from trachea-bronchial regions of human lung tissue.

Air-Liquid Interface (ALI) cultures: alternative culturing platform

Differentiated air-liquid interface (ALI) cultures—tracheo-bronchial ALI cultures. Tracheo-bronchial epithelial cells are isolated as above and expanded in 2D culture on collagen-coated plates. After expansion to P1-5 these cells can either be cryobanked or seeded (4×10⁴-1×10⁵) into collagen-coated TransWells to prepare ALI cultures.

Differentiated air-liquid interface (ALI) cultures—small airway ALI cultures. Methods have been developed for the enrichment of small airway epithelium from dissociated distal lung tissue. Work is in progress to develop and validate small airway ALI cultures.

### Technical Information

### 3D Organoid Culture:

Regional lung organoid models—distal human lung organoids: Inventors are using unfractionated distal lung epithelial cells. Distal lung epithelial cells are recovered from dissociated tissue by FACS, selecting viable cells based upon exclusion of the DNA dye propidium iodide and subsequently using depletion of CD45 and CD31 positive cells and positive selection of CD326 positive cells for enrichment of the epithelial fraction. The Inventors have the potential to further fractionate this population of epithelial cells into alveolar and small airway, based upon either positive or negative selection of cells with the HTII-280 monoclonal antibody. With sufficient tissue and isolated cells this would have been the Inventors' preferred approach as it would allow modeling of both alveolar and small airway compartments. For each organoid culture well, distal lung epithelial cells are then mixed with culture expanded MRCS lung fibroblasts (5×10³ epithelial cells, 7.5×10⁴ MRCS cells) in a total volume of 100 ul and 50 ul of growth factor depleted MatriGel added prior to mixing and placing in a 6.5 mm TransWell. Organoids are cultured for 7-10 days in expansion medium (Pneumacult ALI medium supplemented with a Wnt agonist [CHIR] and Tgf-beta inhibitor [SB431542]) prior to differentiating in the same medium lacking either Wnt agonist or Tgf-beta inhibitor for 4-7 days—medium is placed in the lower compartment of TransWell cultures with the upper surface of polymerized MatriGel at the air interface. These cultures will be infected with a virus by topical application to the culture inserts harboring organoids.

Regional lung organoid models—proximal human lung organoids: These are prepared as above but using epithelial cells isolated from trachea-bronchial regions of human lung tissue. Infection with a virus will be performed as above.

Air-Liquid Interface (ALI) cultures: culture methods themselves have been established in the literature.

Differentiated air-liquid interface (ALI) cultures—tracheo-bronchial ALI cultures. Tracheo-bronchial epithelial cells are isolated as above and expanded in 2D culture on collagen-coated plates. After expansion to P1-5 these cells can either be cryobanked or seeded (4×10⁴-1×10⁵) into collagen-coated TransWells to prepare ALI cultures. The seeded epithelial cells will be allowed to expand to confluence in submerged cultures until confluence using the Pneumacult Ex or Ex-plus media supplemented with hydrocortisone and Rho kinase inhibitor, Y27632, after which time media are removed from the apical compartment of cultures. The media in the basal compartment is changed to Pneumacult ALI media supplemented with hydrocortisone and heparin and the epithelial cells are allowed to differentiate at the air interface to yield a mature pseudostratified epithelium (typically after 14-28 days of culture at air interface). Experimental infection with a virus will be accomplished by application of virus to either apical or basolateral surfaces of ALI cultures.

Differentiated air-liquid interface (ALI) cultures—small airway ALI cultures. In the process. These culture models are being developed and validated. They are being developed from distal lung HTII-280-negative cells.

### Example 11

### Cell Isolation

Human lung tissue was obtained from deceased organ donors in compliance with consent procedures developed by International Institute for the Advancement of Medicine (IIAM) and approved by the Internal Review Board at Cedar-Sinai Medical Center.

Human lung tissue was processed as described previously with the following modifications. For isolation of proximal airway cells, trachea and the first 2-3 generation of bronchi were slit vertically and enzymatically digested with Liberase (50 μg/mL) and DNase 1 (25 μg/mL) incubated at 37° C. with mechanical agitation for 20 minutes, followed by gentle scraping of epithelial cells from the basement membrane. The remaining tissue was finely minced and further digested for 40 minutes at 37° C. For distal alveolar cell isolation, small airways of 2 mm diameter or less and surrounding parenchymal tissue was minced finely and enzymatically digested for 40-60 minutes as described before. Total proximal or distal dissociated cells were passed through a series of cell strainers of decreasing pore sizes from 500 μm to 40 μm under vacuum pressure and depleted of immune and endothelial cells by magnetic associated cell sorting (MACS) in accordance to the manufacturing protocol (Miltenyi Biotec). Viable epithelial cells were further enriched by fluorescence associated cell sorting (FACS) using DAPI (Thermo Fisher Scientific) and antibodies against EPCAM (CD326), CD45 and CD31 (Biolegend) on a BD Influx cell sorter (Becton Dickinson).

### Example 12

### Culture and Differentiation of Proximal Airway Epithelial Cells at Air Liquid Interface

FACS enriched proximal airway epithelial cells were expanded in T25 or T75 flasks coated with bovine type I collagen (Purecol, Advanced biomatrix) in Pneumacult Ex media (STEMCELL Technologies), supplemented with 1× Penicillin-Streptomycin-Neomycin (PSN) Antibiotic Mixture (Thermo Fisher Scientific) and 10 μM Rho kinase inhibitor, Y-27632 (STEMCELL technologies). Upon confluence cells were dissociated using 0.05% Trypsin-EDTA (Thermo Fisher Scientific) and seeded onto collagen coated 0.4 μm pore size transparent cell culture inserts in a 24-well supported format (Corning) at a density of 7.5×10⁴ cells per insert. Cells were initially cultured submerged with 300 μL of Pneumacult Ex media in the apical chamber and 700 μL in the basement chamber for 3-5 days. Upon confluence, cells were cultured at air liquid interface in 700 μL Pneumacult ALT media (STEMCELL Technologies) supplemented with 1×PSN and media was changed every 48 hrs. Cultures were maintained at 37° C. in a humidified incubator (5% CO²) and used for virus infection after 16-20 days of differentiation.

### Example 13

### Culture of 3D Alveolar Organoids

Five thousand FACS enriched distal lung epithelial cells were mixed with 7.5×10⁴ MRCS human lung fibroblast cells (ATCC CCL-171) and resuspended in a 50:50 (v/v) ratio of ice cold Matrigel® (Corning) and Pneumacult ALT medium. 100 uL of the suspension was seeded onto the apical surface of a 0.4 μm pore-size cell culture insert in a 24 well supported format. After polymerization of Matrigel®, 700 μL of Pneumacult ALT medium was added to the basement membrane. Media was supplemented with 50 μg per ml of Gentamycin (Sigma Aldrich) for the first 24 hrs. and 10 μM Rho kinase inhibitor for the first 48 hrs. 2 μM of the Wnt pathway activator, CHTR-99021 (STEMCELL technologies) was added to the media at 48 hrs and maintained for the entire duration of culture. Media was changed every 48 hrs. Cultures were maintained at 37° C. in a humidified incubator (5% CO₂) and used for virus infection after 15 days.

### Additional Representative Embodiments

A method, comprising: providing a quantity of lung tissue; dicing the lung tissue; washing the diced lung tissue; mincing the washed lung tissue; digesting the minced lung tissue with an enzyme.

The method of the preceding paragraph, wherein the lung tissue is distal lung tissue.

The method of a preceding paragraph, wherein dicing the lung tissue generates pieces of about 1 cm³ volume.

The method of a preceding paragraph, wherein washing the dice lung tissue is in HBSS.

The method of a preceding paragraph, wherein mincing the washed lung tissue generate pieces of about 2 mm diameter.

The method of a preceding paragraph, wherein digesting the minced lung tissue with an enzyme is with Liberase™ and DNase.

The method of a preceding paragraph, comprising dissociating digested lung tissue into single cells.

The method of a preceding paragraph, comprising selecting a population of cells.

The method of a preceding paragraph, wherein the population of cells are epithelial cells.

The method of a preceding paragraph, wherein the epithelial cells have a surface marker profile that is one or more of: CD45-negative, CD31-negative, CD236-, DAPI-, and HTII-280-positive.

A method, comprising: providing a quantity of lung tissue; digesting the lung tissue with an enzyme; and dissociating digested lung tissue into single cells.

The method of the preceding paragraph, wherein the lung tissue is proximal lung tissue.

The method of a preceding paragraph, wherein digesting the lung tissue with an enzyme is with Liberase™.

The method of a preceding paragraph, wherein digested lung tissue is further digested with DNAse.

The method of a preceding paragraph, comprising selecting a population of cells.

The method of a preceding paragraph, wherein the population of cells are epithelial cells.

The method of a preceding paragraph, wherein the epithelial cells have a surface marker profile that is one or more of: CD45-negative, CD31-negative, CD236-, DAPI-, HTII-280-positive and NGFR-positive.

A quantity of cells made by any of the methods of the preceding paragraphs.

A cryopreserved quantity of cells made by any of the methods of the preceding paragraphs.

A method of generating lung organoids, comprising: providing a quantity of cells by any of the methods of the preceding paragraphs; culturing the cells in a fluidic device in the presence of a growth media comprising a Rho kinase inhibitor; and further culturing the cells in the presence of a TGFβ inhibitor to generate lung organoids.

The method of a preceding paragraph, wherein the Rho kinase inhibitor is Y-27632.

The method of a preceding paragraph, wherein the TGFβ inhibitor is SB431542.

The method of a preceding paragraph, wherein the fluidic device is a transwell system.

The method of a preceding paragraph, wherein the fluidic device is a microfluidic device.

The method of a preceding paragraph, wherein culturing the cells is for a period of about 7-40 days.

The method of a preceding paragraph, wherein further culturing the cells is for a period of about 15 days.

A quantity of lung organoids made by the method of a preceding paragraph.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”.

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of processing and optionally freezing lung tissues, cells or both, comprising:
cutting lung tissue into about 0.5 cm³ to 2.0 cm³ pieces;
washing the pieces of tissue to remove blood, epithelial lining fluid, or both;
optionally drying the pieces of lung tissue;
removing visceral pleura from the pieces of lung tissue; and
further cutting the pieces of lung tissue into about 0.5-5.0 mm diameter pieces,
wherein the lung tissue is the proximal region of the lung or the distal region of the lung.

2. The method of claim 1, further comprising identifying and separating the proximal and distal regions before cutting the tissue into about 0.5 cm³ to 2.0 cm³ pieces.

3. The method of claim 1, comprising freezing the lung tissues, the method further comprising:
placing the about 0.5-5.0 mm diameter pieces of lung tissue into a vial and cryoprotective media; and
freezing the vial comprising the 0.5-5.0 mm diameter pieces of lung tissue and cryoprotective media to a temperature of about −90 to −70 degrees C.

4. A method of claim 1, further comprising freezing the 0.5-5.0 mm diameter pieces of lung tissue in vapor phase of a liquid nitrogen vessel.

5. A method of claim 1,
wherein cutting lung tissue into about 0.5 cm³ to 2.0 cm³ pieces comprising cutting lung tissue into about 1.0 cm³ pieces, or
wherein further cutting the lung tissue into about 0.5-5.0 mm diameter pieces comprising cutting the lung tissue into about 2-5 mm diameter pieces, or
wherein cutting the lung tissue into about 3-4 mm diameter pieces, or
wherein further cutting the lung tissue into about 0.5-5.0 mm diameter pieces comprising cutting the lung tissue into about 3-4 mm diameter pieces.

6. (canceled)

7. (canceled)

8. A method of claim 1, wherein freezing the vial comprising the pieces of lung tissue to a temperature of about −90 to −70 degrees comprises freezing the vial to about −80 degrees C.

9. A method of enrichment and optionally sub setting of small airway and aveolar epithelial progenitor cells from distal lung tissue, comprising:
performing a method of claim 1, wherein the lung tissue is distal lung tissue, and further cutting the lung tissue into about 0.5-5.0 mm diameter pieces comprises cutting the lung tissue into about 0.5-1.5 mm diameter pieces;
digesting the about 0.5-1.5 mm diameter pieces of lung tissue with enzyme;
dissociating the digested pieces of lung tissue into single cells; and
selecting epithelial progenitor cells.

10. The method of claim 9, wherein cutting the lung tissue into about 0.5-1.5 mm diameter pieces comprises first cutting the 0.5-5.0 mm diameter pieces of lung tissue into about 1.5-2.5 mm diameter pieces, and then cutting the 1.5-2.5 mm diameter pieces into about 0.5-1.5 mm diameter pieces.

11. The method of claim 10, wherein cutting the 1.5-2.5 mm diameter pieces into about 0.5-1.5 mm diameter pieces comprise cutting the 1.5-2.5 mm diameter pieces into about 1.0 mm diameter pieces.

12. A method of claim 9,
wherein the enzyme comprises collagenase I, collagenase II, a non-clostridial neutral protease, or DNase, or combinations thereof, or
wherein selecting the epithelial progenitor cells comprising selecting cells having a surface marker profile that is one or more of: CD45-negative, CD31-negative, and CD236-positive, and optionally have a negative staining for DAPI, or
wherein selecting epithelial progenitor cells comprising depleting immune cells and endothelial cells, cell surface staining for Fluorescence associated cell sorting (FACS), or both.

13. (canceled)

14. A method of claim 9, further comprising sub setting of small airway and aveolar epithelial progenitor cells, the method comprising:
selecting epithelial cells that are HTII-280-negative as small airway epithelial progenitor cells; OR
selecting epithelial cells are HTII-280-positive as alveolar type 2 (AT2) progenitor cells.

15. (canceled)

16. A method of enrichment and optionally subsetting of epithelial progenitor cells from trachea-bronchial airways, comprising:
performing a method of claim 1, wherein the lung tissue is the proximal region of the lung, and wherein luminal epithelial cells have been removed from the proximal region of the lung;
digesting the pieces of lung tissue with enzyme;
dissociating the digested pieces of lung tissue into single cells; and
selecting epithelial progenitor cells.

17. The method of claim 16, further comprising:
performing the following steps before performing a method of claim 1:
open airways of the distal lung tissue along their length to expose their lumen and cover the tissue with a solution comprising collagenase I, collagenase II, a non-clostridial neutral protease, or a combination thereof;
stripping the luminal epithelial cells from the tissue; and
collect the luminal epithelial cells.

18. A method of claim 16, further comprising dissociating luminal epithelial cells into single cells.

19. A method of claim 16, further comprising dissociating luminal epithelial cells into single cell luminal epithelial cells and combining the single cell luminal epithelial cells with the single cells of claim 16 before selecting for epithelial progenitor cells.

20. A method of claim 16,
wherein the enzyme comprises collagenase I, collagenase II, a non-clostridial neutral protease, or DNase, or combinations thereof, or
wherein selecting the epithelial progenitor cells comprising selecting cells having a surface marker profile that is one or more of: CD45-negative, CD31-negative, and CD236-positive, and optionally have a negative staining for DAPI.

21. (canceled)

22. A method of claim 16, further comprising sub setting of epithelial progenitor cells, the method comprising:
selecting epithelial progenitor cells that are NGRF-positive as a basal cell type; OR
selecting epithelial progenitor cells that are NGRF-negative as a non-basal cell type.

23. A method of claim 16, wherein selecting epithelial progenitor cells comprising depleting immune cells and endothelial cells, cell surface staining for fluorescence associated cell sorting (FACS), or both.

24. A method of generating a lung organoid, comprising:
providing a quantity of cells of claim 1;
culturing the cells in the presence of a growth media comprising a Rho kinase inhibitor; and
further culturing the cells in the presence of a TGFβ inhibitor to generate lung organoids.

25. The method of claim 24,
wherein the Rho kinase inhibitor is Y-27632 or
wherein the TGFβ inhibitor is SB431542, or
wherein the fluidic device is a transwell system, or
wherein the fluidic device is a microfluidic device, or
culturing the cells is for a period of about 7-40 days, or
wherein further culturing the cells is for a period of about 15 days.

26. (canceled)

27. (canceled)

28. (canceled)

29. (canceled)

30. (canceled)

31. A quantity of lung organoids made by a method of claim 24.

32. A system for modeling a lung, comprising:
a population of cells comprising cells selected from the group consisting of lung cells isolated by the method of claim 1, primary lung cells differentiated from lung cells isolated by the method of claim 1, a lung organoid comprising the lung cells or the primary lung cells; and
a cell culture device, a cell culture plate, or a multi-well culture plate, optionally, wherein the population of cells are in the cell culture device, the cell culture plate, or the multi-well culture plate.

33. (canceled)

34. A system for test agent screening in a lung model, comprising:
a population of cells comprising cells selected from the group consisting of lung cells isolated by the method of claim 1, primary lung cells differentiated from lung cells isolated by the method of claim 1, a lung organoid comprising the lung cells or the primary lung cells; and
a cell culture device, a cell culture plate, or a multi-well culture plate;
wherein the test agent and the population of cells, are in contact in the cell culture plate, or the multi-well culture plate.

35. The system of claim 33, wherein cell culture device is an air-liquid interface culture or a Transwell system comprising the population of cells.

36. A system of claim 32, wherein the lung cells are epithelial cells.

37. The system of claim 36, wherein the epithelial cells are small airway epithelial progenitor cells, alveolar type 2 (AT2) progenitor cells, basal cell type, or non-basal cell type, or wherein the epithelial cells are proximal airway cells, or distal alveolar cells.

38. (canceled)

39. A method selecting an agent of interest, comprising:
contacting a test agent with a population of cells comprising cells selected from the group consisting of lung cells isolated by the method of claim 1, primary lung cells differentiated from lung cells isolated by the method of claim 1, a lung organoid comprising the lung cells or the primary lung cells, wherein the test agent and the population of cells are in contact in a cell culture device, a cell culture plate, or a multi-well culture plate;
measuring a parameter in the population of cells; and
selecting the test agent as the agent of interest based on the measured parameter in the population of cells.

40. A method modeling a lung, comprising:
measuring a parameter in a population of cells comprising cells selected from the group consisting of lung cells isolated by the method of claim 1, primary lung cells differentiated from lung cells isolated by the method of claim 1, a lung organoid comprising the lung cells or the primary lung cells, wherein the population of cells are in contact in a cell culture device, a cell culture plate, or a multi-well culture plate, optionally further comprising contacting a test agent to the population of cells before, while, after or a combination thereof, measuring the parameter.

41. (canceled)

42. A method of claim 39, wherein the parameter comprises a phenotype of interest, an expression level of a gene, or an expression level of a protein of interest, or combination thereof.

43. The method of claim 39, wherein cell culture device is an air-liquid interface culture or a Transwell system comprising the population of cells.

44. A method of claim 39,
wherein the lung cells are epithelial cells,
wherein the lung cells are fibrotic lung cells, or
wherein the lung cells are idiopathic fibrotic lung cells.

45. The method of claim 44, wherein the epithelial cells are small airway epithelial progenitor cells, alveolar type 2 (AT2) progenitor cells, basal cell type, or non-basal cell type, or wherein the epithelial cells are proximal airway cells, or distal alveolar cells.

46. (canceled)

47. (canceled)

48. (canceled)
